(19) United States
(12) Patent Application Publication
(10) Pub. No.: US 2016/0215111 A1
(43) Pub. Date: Jul. 28, 2016

(54) MATERIALS AND METHODS

(71) Applicant: THE UNIVERSITY OF SYDNEY, Sydney, New South Wales (AU)

(72) Inventors: Marcela Bilek, Sutherland, New South Wales (AU); Aleksey Kondyurin, Penrith, New South Wales (AU); Daniel Victor BAX, Maroubra, New South Wales (AU); Anthony Steven Weiss, Randwick, New South Wales (AU)

(73) Assignee: THE UNIVERSITY OF SYDNEY, Sydney, New South Wales (AU)

(21) Appl. No.: 14/915,933

(22) PCT Filed: Sep. 2, 2014
(86) PCT No.: PCT/AU2014/050204

§ 371 Date: Mar. 2, 2016

(30) Foreign Application Priority Data
Sep. 2, 2013 (AU) 2013903334

(51) Int. Cl. C08J 7/12; A61L 31/10; A61L 31/16; A61L 31/04

(52) U.S. Cl. CPC C08J 7/123; A61L 31/048; A61L 31/10; A61L 31/16; C08J 2325/06; C08J 2327/18; C08J 2489/00; A61L 2400/18; A61L 2420/00; A61L 2400/02; A61L 2300/25

(57) ABSTRACT

The invention relates to methods of controlling orientation of direct covalent binding of a peptide to a polymer substrate surface, to surfaces with peptides directly covalently bound thereto in a manner where the orientation of binding is controlled as well as to devices comprising such substrates. In particular the invention relates to A method of controlling predominant orientation of direct covalent binding of one or more peptides to a polymer substrate surface comprising: (a) exposing the surface to energetic ion treatment to generate a plurality of activated sites comprising reactive radical species; (b) incubating the surface with one or more peptide/s that exhibit or can be induced to exhibit a dipole moment and manipulating the electric field environment and/or charge of said surface and/or of said peptide/s during said incubating; wherein predominant orientation of direct covalent binding of said peptide/s to said surface is thereby controlled.

### FIELD OF THE INVENTION

The present invention relates in particular, but not exclusively, to methods of controlling orientation of direct covalent binding of a peptide to a polymer substrate surface, to surfaces with peptides directly covalently bound thereto in a manner where the orientation of binding is controlled as well as to de ices comprising such substrates.

### BACKGROUND OF THE INVENTION

The advent of diagnostic array technology (where for example protein, antibody or other biological molecule/s is/are attached at discrete locations on a substrate surface to allow attachment of other molecules of interest (target molecules) and where means for detecting the attachment of the target molecules is provided) has led to an increased demand for surfaces capable of binding to biological molecules such as antibodies, peptides/proteins, nucleic acids and cells. It is similarly necessary in other applications, such as for example biosensors, medical devices where biocompatible surfaces are required and in the screening of active agents against drug targets, that surfaces capable of binding to biological molecules are required.

An ideal surface for these applications should bind proteins or other biological molecules while preserving their functionality. The binding is preferably strong and stable over extended periods to allow repeated washing steps during processing. In many of these technologies the protein (or other biological molecule) binding to the substrate surface is attached through non-specific physisorption, leading to losses of protein during washing and variability in the degree of attachment given that the attachment process is molecular species dependent. Functionality of physisorbed proteins depends strongly on the energetics of the interaction with the surface and will vary across proteins.

It is of interest to be able to attach biological molecules strongly, preferably by means of a covalent bond, to surfaces of metals, semiconductors, polymers, composite materials and/or ceramics in a variety of applications. For example, metals have desirable strength and elastic properties that make them suitable for use in repairing human and animal bones and joints. In particular, metal prosthetic pins and plates can be used to repair bone after fracture. In this context it is desirable to attach bone cells firmly to the metal surface so that the metal part is firmly anchored in the skeleton For such applications it is desirable to promote the healthy growth of oesteoblasts and to suppress growth of fibroblasts that give rise to fibrous tissue. Such differentiation of cell attachment can be facilitated by attaching to the surface one or more suitable biologically active molecules. Another application of a metal prosthetic part is in stents for maintaining flow through blood vessels or other body cavities. Such devices should be biocompatible but should not promote excessive fibrous tissue or smooth muscle cell growth, whilst promoting the attachment and growth of endothelial cells. Such differentiation can also be attained by attaching suitable biological molecules to the metal surface.

It is also desirable to be able to covalently attach biological molecules to the surfaces of ceramics for purposes of skeletal repair, for the same reasons as outlined above in relation to metals. Indeed there are a variety of other contexts in which it is desirable to be able to covalently attach biological molecules to the surfaces of metals, ceramics, semiconductors, polymers or to the surfaces of composite materials that have some metallic, ceramic, polymeric and/or semiconductor characteristics or features. For example, it is desirable to attach biological molecules to surfaces in the contexts of assays and detection devices, scaffolds for tissue and/or organ generation, screening of compounds for useful biological activity, micro- and nano-devices that interact with or include biological components (e.g. molecular motors involving actin/myosin filaments), fuel cells that incorporate a biological processing component (e.g. fuel cells comprising photosynthetic cells). A further specific example is that of semiconductors that can be used for the detection of biological molecules by sensing the specific attachment of the target molecules to detection molecules bound on the semiconductor surface.

In earlier work by the present research group (see International patent publication nos. WO2007/104107 and WO2009/015420) methods have been devised to covalently bind functional biological molecules to a polymer substrate and to other substrates such as metal, semiconductor, ceramic or composite substrates, without the need to use linker molecules (and therefore without associated wet chemistry). In one aspect the earlier work involves a two step plasma modification process including ion implantation and/or deposition, to create a mixed or graded interface, followed by the deposition of a hydrophilic plasma polymer. The binding of biological molecules then involves simple adsorption (resulting in covalent binding), with no further chemistry required, and the surfaces produced retain the biological functionality of the bound biological material.

The present inventors have now devised a means of covalently binding functional peptides to a polymer substrate surface wherein the orientation of binding of the peptide can be controlled. This technique is particularly useful as it enables the ability to expose, or for that matter hide, a particular functional site or epitope of the peptide bound to the surface so that it is either available or not available, as required, for interaction with other molecules or agents. For example, a surface can be prepared using techniques of the present invention wherein cell ligand or antibody binding peptides are covalently bound to the surface in a manner that enables binding to the corresponding cell, ligand and/or antibody. Alternatively, the surface can be prepared using the techniques of the invention to disable the interaction, between the bound peptide and its binding partner cell, ligand and/or antibody. The surfaces can also be prepared such that they are patterned to include areas where binding of peptide to the surface is enabled or inhibited and/or areas where binding between peptide and its binding partner is enabled and areas where binding between peptide and its binding partner is not enabled. The orientation of binding of a particular peptide to a surface can be altered or switched by modifying incubation conditions and location of binding of peptides to the surface can be controlled by masking the surface.

### SUMMARY OF THE INVENTION

According to one embodiment of the present invention there is provided a method of controlling predominant orientation of direct covalent binding of one or more peptides to a polymer substrate surface comprising:
(a) exposing the surface to energetic ion treatment to generate a plurality of activated sites comprising reactive radical species;
(b) incubating the surface with one or more peptide/s that exhibit or can be induced to exhibit a dipole moment and manipulating the electric field environment and/or charge of said surface and/or of said peptide/s during said incubating;
   wherein predominant orientation of direct covalent binding of said peptide/s to said surface is thereby controlled.

According to another embodiment of the present invention there is provided a polymer substrate surface having a peptide directly covalently bound thereto in a manner wherein the predominant orientation of binding of the peptide to the surface is controlled, produced by a method as described above.

According to another embodiment of the present invention there is provided a polymer substrate surface having a peptide directly covalently bound thereto in a manner wherein the predominant orientation of binding of the peptide to the surface is controlled.

According to another embodiment of the present invention there is provided a device comprising a polymer substrate surface as described above.

### BRIEF DESCRIPTION OF THE FIGURES

The invention will be further described with reference to the figures:

FIG. 1: A) Schematic showing the amino acid sequence of peptide 36 and ΔRKRK peptide 36. B) Percentage cell attachment to untreated polystyrene (gray crosses, dashed line) or PIII treated polystyrene (black diamond, solid line) coated with an increasing concentration of peptide 36. Samples were BSA blocked. Error bars indicate standard deviations of triplicate measurements.

FIG. 2: ATR-FTIR detection of peptide 36 bound to PIII treated (spectra a and c) or untreated (spectra b and d) polystyrene. Samples were measured before (a and b) or after (c and d) Tween-20 washing. No-peptide control spectra were subtracted to remove polymer associated peaks. Peptide associated amide peaks at ˜3300 nm, ˜1650 nm and ˜1550 nm on spectra a, b and c are indicated with arrows.

FIG. 3: A, B) Percentage cell attachment to untreated (gray bars) or PIII treated (black bars) polystyrene. The peptide, BSA blocking and Tween-20 washing conditions are stated under each set of bars. Error bars indicate standard deviations of triplicate measurements. C) Phase contrast microscopy of cells adherent to untreated or PIII treated polystyrene coated with peptide 36 or ΔRKRK peptide 36. Scale bars indicate 100 μm. D) Schematic representation of peptide 36 orientation on untreated (left) or PIII treated (right) polymers. The cell adhesive RKRK motif is shown in red.

FIG. 4: Phase contrast microscopy at 10× magnification (left) and 20× magnification (right) of cells adherent to contact masked PIII treated polystyrene (A) and shadow masked PIII treated PTFE (B). The peptide coating conditions are stated to the left. PIII treated (PIII) and masked (UT) regions are denoted and the boundary between the PITT treated and masked regions is shown by a dashed line. Scale bars indicate 200 μm.

FIG. 5: A) Toluidine blue O detection of negatively charged COOH groups on PTFE surfaces treated with increasing durations of N₂ plasma. Unstained PTFE controls were subtracted from the readings. The surfaces were not coated with peptide 36. B) Percentage cell attachment to peptide 36 coated PTFE. The PTFE was treated with increasing durations of N₂ plasma prior to peptide 36 coating. Samples were BSA blocked to prevent non-specific cell binding to the PTFE surfaces. Cell attachment to peptide 36 coated PTFE treated with PIII for 800 seconds is shown with a dashed line. C) Overlay of % cell attachment to bound peptide 36 (from (B)) and toluidine blue O detection of surface COOH groups (from (A)) with increasing N₂ plasma treatment of the underlying PTFE surface. A linear relationship between toluidine blue O detection and cell attachment to bound peptide 36 is shown inset. Error bars indicate standard deviations of triplicate measurements.

FIG. 6: A) Orange II dye detection of positively charged C═NH and C—NH₂ groups on PTFE surfaces exposed to allylamine vapors for increasing durations post PIII treatment. B). Percentage cell attachment to untreated (white bars), PIII treated (gray bars) and PIII treated then allylamine vapor exposed (30 min exposure, black bars) PTFE surfaces. Peptide coating and BSA blocking conditions are stated under each set of bars. Error bars indicate standard deviations of triplicate measurements.

FIG. 7: A, B) Percentage cell attachment to untreated (crosses, gray dashed line) or PIII treated (diamonds, black solid line) polystyrene A) coated with 50 μM peptide 36 in 10 mM PO₄ buffer containing increasing NaCl molarity. The pH was maintained at pH 7.4. The red data points indicate 150 mM NaCl which corresponds to standard PBS conditions used for the previous assays. B) coated with 50 μM peptide 36 in 10 mM PO₄ buffer of increasing pH. The NaCl concentration was maintained at 150 mM. For (A, B) all samples were BSA blocked to prevent non-specific cell binding to the polystyrene surfaces. C, D) Percentage cell attachment to untreated (gray bars) or PIII treated (black bars) PTFE (C) or PEEK (D). The peptide coating, buffer pH and BSA blocking are stated under each set of bars. For all graphs buffer conditions denote the peptide binding buffer; the samples were returned to standard PBS conditions (10 mM PO₄, 150 mM NaCl, pH 7.4) immediately post peptide binding. Error bars indicate standard deviations of triplicate measurements.

FIG. 8: A, B) C-terminal ELISA detection of peptide 36 RKRK exposure on untreated (cross, gray dashed line) or PIII treated (diamond, black solid line) polystyrene A) from an increasing coating concentration of peptide 36. The coating buffer was held constant at 10 mM PO₄, 150 mM NaCl, pH 7.4. B) coated with 50 μM peptide 36 in 10 mM PO₄ buffer of increasing pH. The NaCl concentration was maintained at 150 mM. C) Overlay of % cell attachment to peptide 36 (from FIG. 7B) and C-terminal ELISA detection of RKRK exposure of peptide 36 (from FIG. 8B) from a buffer of increasing pH (50 μM peptide 36, 10 mM PO₄, 150 mM NaCl). A linear relationship between ELISA detection of exposed RKRK motifs and cell attachment to bound peptide 36 is shown inset. Error bars indicate standard deviations of triplicate measurements.

FIG. 9: Percentage cell attachment to untreated (crosses, gray dashed line) or PIII treated (diamonds, black solid line) PTFE. Samples were coated with 50 μM peptide 36 in 10 mM PO₄, 0 mM NaCl, pH 10 buffer then heated for 10 min at increasing temperatures in 10 mM PO₄, 150 mM NaCl, pH 7.4 buffer. All samples were BSA blocked to prevent non-specific cell binding to the PTFE surfaces. Error bars indicate standard deviations of triplicate measurements.

FIG. 10: Percentage cell attachment to untreated or PIII treated PTFE. All samples were coated with 50 μM peptide 36 in 10 mM PO₄, 0 mM NaCl, pH 10 buffer then autoclaved in a dry beaker (dry autoclave, dark gray bars) or in 10 ml of buffer (liquid autoclave, black bars). Liquid autoclave was conducted in A) 10 mM PO₄, 150 mM NaCl, pH 6 or B, C) 10 mM PO₄, 0 mM NaCl, pH 10. For A, B) the samples were removed from the autoclave liquid once it had cooled to 70° C. and placed in room temperature PBS. For C) the samples were allowed to cool in the autoclave liquid to room temperature for 4 hours. Samples not subjected to autoclaving are shown in light grey. All samples were BSA blocked to prevent non-specific cell binding to the PTFE surfaces. Error bars indicate standard deviations of triplicate measurements.

FIG. 11: Microscopy of cells adherent to tape masked, PIII treated PTFE. The peptide 36 (50 μM) association and dissociation conditions are detailed above each photograph. PIII treated (PIII) and masked, untreated (UT) regions are labelled and the boundary between the PIII treated and masked regions is denoted by a dashed line. Scale bars indicate 100 μm.

FIG. 12: Model for the charged based modulation of the cell adhesive properties of peptide 36 (green). Untreated polymer (blue) is uncharged and so the peptide is not electrostatically orientated, resulting in solvent exposure and so integrin-mediated cell attachment to the RKRK motif. In contrast PIII treated polymer surfaces (gray) possess negatively charged COOH groups which interact with the positively charged RKRK motif, hindering integrin accessibility and so reducing cell binding.

FIG. 13: A, B) C-terminal ELISA detection of RKRK exposure on tissue culture plastic of A) peptide 36 (cross, solid line) and ΔRKRK peptide 36 (diamond, dashed line) bound from an increasing coating concentration. B) WT tropoelastin (cross, solid line) and ΔRKRK tropoelastin (square, gray dashed line) bound from an increasing coating concentration. The positive dashed line indicates the absorbance from wells in which the primary antibody was added without pre coating with tropoelastin/peptide or BSA block. Error bars indicate standard deviations of triplicate measurements.

FIG. 14: Percentage cell attachment to 50 μM peptide 36 coated untreated PTFE, freshly generated PIII treated PTFE or PIII treated PTFE after storage at room temperature for 1 year. Peptide 36 was coated in 10 mM PO₄, 150 mM NaCl, pH 7.4. BSA blocking was used to prevent non-specific cell binding to the PTFE surfaces. Error bars indicate standard deviations of triplicate measurements.

### DETAILED DESCRIPTION OF THE INVENTION

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Documents referred to within this specification are included herein in their entirety by way of reference.

The reference to any prior art in this specification is not, and should not be taken as, an acknowledgment or any form of suggestion that that prior art forms part of the common general knowledge in Australia.

As mentioned above, in one broad embodiment this invention relates to a method of controlling predominant orientation of direct covalent binding of one or more peptides to a substrate surface. In a broad aspect the method involves steps of:
(a) exposing the surface to energetic ion treatment to generate a plurality of activated sites comprising reactive radical species;
(b) incubating the surface resulting front step (a) with one or more peptide/s that exhibit or can be induced to exhibit a dipole moment and manipulating the electric field environment and/or charge of the surface and/or of the peptide/s during the incubating; with the result that the predominant orientation of direct covalent binding of the peptides to the surface is thereby controlled.

The present inventors understand that through the activation of a polymer surface layer, including a plasma polymer surface layer, on a substrate (such as a metal, semiconductor, polymer, composite and/or ceramic substrate) it is possible to form direct chemical bonds, to chemical groups of peptides. The inventors understand that activation of the polymer surface involves the generation of reactive radicals, which are mobile under the surface and available, upon reaching the surface, as binding sites for reactive species on peptides, such as amine, thiol or carboxyl groups. The inventors believe that in some but not all cases, oxygen plays a role in reacting at the activated sites to generate reactive oxygen species such as charged ester, carbonyl and carboxylic acid moieties that are also available for covalent binding interactions with peptide reactive species. Involvement of oxygen is likely in cases where the activated surface has been exposed to air or an oxygen containing or rich atmosphere during or after activation of the polymer surface. However, given the difficulty of excluding all oxygen even when the surface is treated in an atmosphere that supposedly excludes oxygen (such as under nitrogen or argon), and because of the high reactivity of oxygen, oxygen may also play a role in generating reactive oxygen species under such conditions.

Without wishing to be bound by theory, the present inventors understand that their technique for controlling the orientation or directionality of binding of a peptide to a polymer surface operates by influencing the orientation at which peptides approach a surface that has been activated for covalent binding to the surface, utilising electrostatic interactions between charged chemical groups on the surface and the peptide or applied electric fields as the mechanism for exerting orientation control. It will be well understood by persons skilled in the art, in view of this mechanistic background, that orientation control will be exerted on a mass scale across the surface concerned that results from adoption of the lowest energy state for given binding interactions subject to thermal fluctuations. Therefore, whilst the most energetically favourable binding orientation will predominate, this will not preclude a proportion of peptides adopting alternate binding configurations. However, the ensemble average will be driven towards the desired binding configuration as the desired configuration is made more energetically favourable under the conditions adopted—for example by adopting more extreme ionic strength, pH or externally applied current or voltage conditions to influence the electrostatic forces at the substrate surface. Although in a strict sense it is unlikely that all peptides bound to the surface will bind in the desired orientation a high proportion of binding in the desired orientation can be achieved by adopting the appropriate conditions for manipulating charge of the species on the substrate surface and/or of the peptide/s and/or by manipulating the electric field environment during the incubating step when the peptides are exposed to the activated surface. Once the peptides become covalently bound this orientation will be locked in regardless of further changes in the environment. For example at least about 70%, such as at least about 75%, at least about 80%, at least about 85%, at least about 90%, at least about 95%, at least about 97%, about 98%, about 99%, about 99.5% or about 99.9% binding in the desired orientation can be achieved by adopting appropriate conditions. In some embodiments of the invention the predominant orientation of direct covalent binding is controlled by in fact favouring mixed or random orientation. In this aspect of the invention a peptide that exhibits a dipole moment under some conditions is induced to exhibit no effective dipole moment as a result of manipulation of the electric field environment and/or charge during incubation, such that whereas under one set of conditions (such as at physiological pH) there would be binding in one predominant orientation, the predominant orientation is controlled by preventing this orientation and encouraging an essentially random binding orientation. Therefore, the desired orientation may in fact be a mixed binding orientation in the case where a mixed binding orientation would not be favoured when manipulation of the electric field environment and/or charge is not performed.

By the term “activated” it is intended to mean that the polymer substrate or polymeric surface layer of the substrate being treated (e.g. metal, semiconductor, polymer, composite and/or ceramic substrate) has been processed by energetic ion treatment such that it is able to accept a biological molecule for binding, upon exposure thereto. That is, the polymeric surface layer on the substrate has one or more higher energy state regions where there are unpaired electrons (reactive radical species) available for participation in binding to a chemical group on a peptide. In one aspect, if there has been exposure to oxygen, the activated surface may include reactive oxygen species as the reactive species that are available for participation in binding to a chemical group on a peptide.

Within this application we refer to binding of a peptide as functionalisation of the polymeric surface on the substrate material and to the polymer surface of the substrate to which the peptide is bound as being “functionalised”. Attachment by covalent bonds to a preferably hydrophilic surface allows strong time stable attachment of peptides that are able to maintain a useful biological function. For example, an hydrophilic polymer surface of the substrate will ensure that it is not energetically favourable for proteins to denature on the surface. Covalent attachment to a surface can be achieved via amino acid side chain groups covalently attached to the surface, for example. The strategy adopted is to prepare the polymer surface, such as a plasma polymer surface, with sites that encourage covalent attachment. In one approach energetic ion bombardment (such as by plasma immersion ion implantation (PIII) or ion beam exposure) of an existing polymer or polymer-coated substrate is utilised to create embedded radicals that give rise to binding sites upon migrating to the surface. Cross-linking created by internal bonding of a portion of the unpaired electrons stabilises the polymer surfaces. Alternatively, in the case of non-polymeric substrates a deposition process with energetic for bombardment can be used to create a polymeric surface layer with embedded, mobile radicals that give rise to binding sites upon migrating to the surface. Cross-linking created by internal bonding of a portion of the unpaired electrons during deposition of the plasma polymer stabilises the surface. Using functionality assays, the inventors have demonstrated that associated with the adopted energetic ion treatment there is enhancement of functional protein attachment with covalent binding, compared to non-treated surfaces, as well as significantly increased resistance to repeated washing steps. That is, the presence of the surface does not induce the proteins or peptides to adopt conformations that are not native in solution, the binding is strong and can withstand repeated washing and the peptide is able to retain useful activity.

Throughout this specification it is to be understood that the term “peptide” is intended to include within its scope any chain of naturally occurring and/or synthetic amino acids, including amino acid sequences that may more conventionally be referred to as proteins due to their length, wherein the peptide exhibits or can be induced to exhibit a dipole moment. For example, the peptides may comprise from about 3 to about 1000, such as from about 5 to about 500, about 7 to about 250, about 9 to about 100 or about 11 to about 50 amino acids in length. In particular aspects of the invention the peptides are from about 3 to about 50 or about 5 to about 25 or about 7 to about 15 amino acids in length. There is advantage in one aspect of the invention in adopting shorter sequence peptides such as those of about 5 to about 25 amino acids in length as surfaces on substrates or devices requiring sterilisation that are functionalised with such short peptides have been shown by the present inventors to be amenable to sterilisation by autoclaving without adversely affecting the structure and therefore biological function of such short peptides. Similarly shorter peptides can be exposed to incubation conditions involving extremes of pH or ionic strength without adverse effect upon the biological activity of the peptide. As longer peptides, including proteins, are adopted it is necessary to reduce the duration and/or severity of incubation conditions to avoid peptide degradation and/or disruption of three dimensional conformation and therefore biological activity.

The peptides utilised in the present invention have the ability to exhibit a dipole moment under specific conditions and are referred to throughout this specification for convenience simply as “peptides”. Dipole moments in peptides arise from asymmetry in the arrangement of amino acids and charges can be induced and varied by alterations in electric field environment and/or charge, such as by varying buffer pH, ionic strength and/or by applying an electric field. Peptides with the ability to exhibit a dipole moment can readily be designed and synthesised by skilled persons by including within the peptide electron withdrawing or donating or charged natural, modified or non-naturally occurring amino acids or other electron withdrawing or donating or charged chemical groups at or towards one or both termini, such that the peptide exhibits a dipole moment under desired conditions. That is, under specific incubation conditions the orientation of the peptide relative to the surface (including a random orientation) at which binding is intended will be controlled as a result of electrostatic forces. Persons skilled in the art would readily recognise, for example, that lysine (Lys) and arginine (Arg) are positively charged at neutral pH and histidine (His) may be positively charged or neutral depending upon its local environment, whereas glutamate (Glu) and aspartate (Asp) are negatively charged at neutral (physiological) pH. With longer peptides (including proteins) the electrostatic asymmetry between the termini that is built into the peptide design may need to be more extreme in order to overcome the increased electrostatic orientation inertia arising from the larger structure.

The term “peptide” also encompasses a combination or mixture of peptides and includes active fragments—that is peptide sequences derived from an active protein that exhibit preferably at least at least 20%, preferably at least 40%, more preferably at least 60%, 70% or 80% and most preferably at least 90%, 95%, 98% or 99% of the activity of the active protein. Examples of what are considered to fall within the ambit of peptides in the context of this invention include, but are not limited to, enzymes, proteins, glycoproteins, lipoproteins, as well as active fragments thereof. For example, such molecules may take the form of antibodies, immunoglobulins, complementarity determining regions, receptors, enzymes, peptide neurotransmitters or other cell signalling agents, cytokines, hormones and active fragments thereof. The term “peptide” also encompasses peptides and proteins that are integral to or attached to cells or cellular components (eg. cell membrane proteins) through which cells or cellular components may be bound to the polymer surface. Further specific examples of peptides included within the invention are peptide or protein toxins and poisons including naturally occurring toxins such as bacterial, viral, plant or animal derived peptide or protein toxins or active fragments thereof including conotoxin and snake and spider venoms, for example. A peptide (protein) of particular interest is tropoelastin, which is an extracellular matrix protein that can be used to functionalise surfaces to improve the biological compatibility of implantable or other devices. Enzymes of interest include those capable of breaking down cellulose into simple sugars such as cellulase. Peptides according to the invention can be produced by convention means such as utilising chemical techniques, automated peptide synthesis apparatus and recombinant DNA techniques in appropriate cell lines, such as for example described in detail in Sambrook and Russell (2001), Molecular Cloning: A Laboratory Manual (3rd ed.). Cold Spring Harbor Laboratory Press. ISBN 978-0-87969-577-4.

By the term “functional” it is intended to convey that the peptide is able to exhibit at least some of the activity it would normally exhibit in a biological system. For example, activity may include the maintained ability to participate in binding interactions, such as antigen/antibody binding, receptor/drug binding, the maintained ability to catalyse or participate in a biological reaction or the ability to interact with cell membrane proteins in biological tissues and to bind to cells even, if this is at a lower level than is usual in a biological system. Routine assays are available to assess functionality of the peptide. Preferably the activity of the peptide bound to the activated polymer surface is at least 20%, preferably at least 40%, more preferably at least 60%, 70% or 80% and most preferably at least 90%, 95%, 98% or 99% of the activity of the peptide when not bound to the surface. Most preferably the activity of the bound peptide is equivalent to that of a non-bound molecule.

An advantage associated with the present invention is that the process for binding peptides to the surface of a metal, semiconductor, polymer, composite and/or ceramic does not depend upon the specific biological molecule or metal, semiconductor, polymer composite and/or ceramic and can therefore be applied to a wide variety of peptides and metals, semiconductors, polymers, composites and/or ceramics. Furthermore, the present invention does not require linker molecules to be utilised, which means that time consuming and potentially costly and complex wet chemistry approaches for linkage are not required and waste associated with solvent disposal is eliminated.

As indicated above the present invention can be utilised to attach functional peptides to surfaces of a wide variety of metal, semiconductor, polymer, composite and/or ceramic substrates, which will be referred to herein simply as “substrates”. For example the substrate may take the form of a block sheet, film, foil, tube, strand, fibre, piece or particle (eg. a nano- or micro-particle such as a nano- or micro-sphere), powder, shaped article, indented, textured or moulded article or woven fabric or massed fibre pressed into a sheet (for example like paper) of metal, semiconductor, polymer, composite and/or ceramic. The substrate can be a solid mono-material, laminated product, hybrid material or alternatively a coating on any type of base material which can be non-metallic or metallic in nature, and which may include a polymer component, such as homo-polymer, co-polymer or polymer mixture. Indeed, the substrate may also form a component of a device, such as for example a component of a diagnostic kit or detection device, a tissue, cell or organ culture scaffold or support, a biosensor, an analytical plate, an assay component, a micro- or nano-device that interacts with or includes biological components (e.g. molecular motors involving actin/myosin filaments) or a medical device such a contact lens, a stent (eg a cardiovascular or gastrointestinal stent), a pace maker, a hearing aid, a prosthesis, an artificial joint, a bone or tissue replacement material, an artificial organ, a heart valve or replacement vessel, a suture, staple, nail, screw, bolt or other device for surgical use or other implantable or biocompatible device.

Other devices that may be produced according to the invention are those related to chemical processing. For example, the invention includes devices utilised in chemical processes conducted on surfaces or substrates that may result in generation of fuels, biofuels, electricity or production of chemical products (e.g. bulk or fine chemicals, drugs, proteins, peptides, nucleic acids, polymers, food supplements and the like). In a preferred embodiment the invention includes devices used in the production of ethanol by the action of enzymes on sugars or cellulose or other agents. The invention also includes devices used in production of electricity by means of a chemical reaction catalysed by an enzyme, such as in a fuel cell or bio-fuel cell and fuel cells or substrates that incorporate a biological processing component (e.g. fuel cells comprising photosynthetic cells). The functionalised substrate can for example form an electrode of such a fuel cell. In this context the invention provides surfaces functionalised by directionally orientated enzymes that can be made available to chemical agents to be processed by immersion in them or by arranging for the agents to flow over the surfaces. In the case that the agent flows over the enzyme-functionalised surface, problems with the poisoning of the enzyme by the products of the reaction can be minimised. Another advantage of the invention is that the enzyme functionalised surface can be rapidly and conveniently replaced with another fresh functionalised surface in the event that the enzymes become poisoned or are otherwise rendered inactive, without the need to dispose of the entire batch of chemicals.

A further specific example of devices of the invention is semiconductors, such as CMOS devices, that can be used for the detection of biological molecules by sensing the specific attachment of the target molecules to detection peptides bound on the semiconductor surface, or that are components of bio-devices including bio-computers.

The substrates of the invention will include a polymer surface thereon, which may take the form of a polymer coating, sheath or covering or alternatively a more integral plasma polymer generated surface layer that may be produced by methods disclosed in the present research group's earlier international patent publication no. WO2009/015420. The term “polymer” as it is used herein is intended to encompass homo-polymers, co-polymers, polymer containing materials, polymer mixtures or blends, such as with other polymers and/or natural and synthetic rubbers, as well as polymer matrix composites, on their own, or alternatively as an integral and surface located component of a multi-layer laminated sandwich comprising other materials e.g. polymers, metals or ceramics (including glass), or a coating (including a partial coating) on any type of substrate material. The term “polymer” encompasses thermoset and/or thermoplastic materials as well as polymers generated by plasma deposition processes. The term “polymer” also encompasses polymer like surfaces that include reactive species or electrons and which may approach, generally or in isolated regions, the appearance and structure of amorphous carbon. The polymer surfaces may fully or partially coat or cover the substrate, may include gaps or apertures and/or regions of varied thickness, where the gaps or apertures and regions of varied thickness may be consistent, ordered, patterned and/or repeated or may be random or disordered.

The polymeric substrates which can be treated according to the present invention include, but are not limited to, polyolefins such as low density polyethylene (LDPE), polypropylene (PP), high density polyethylene (HDPE), ultra high molecular weight polyethylene (UHMWPE), blends of polyolefins with other polymers or rubbers; polyethers such as polyoxymethylene (Acetal), polyamides, such as poly(hexamethylene adipamide) (Nylon 66); polyimides; polycarbonates; halogenated polymers, such as polyvinylidenefluoride (PVDF), polytetra-fluoroethylene (PTFE) (Teflon™), fluorinated ethylene-propylene copolymer (FEP), and polyvinyl chloride (PVC); aromatic polymers, such as polystyrene (PS); ketone polymers such as polyetheretherketone (PEEK); methacrylate polymers, such as polymethylmethacrylate (PMMA); polyesters, such as polyethylene terephthalate (PET); and copolymers, such as ABS and ethylene propylene diene mixture (EPDM). Preferred polymers include polyethylene, PEEK and polystyrene.

Throughout this specification the term “plasma polymer” is intended to encompass a material produced on a surface by deposition from a plasma, into which carbon or carbon containing molecular species are released. The carbon containing molecular species are fragmented in the plasma and a plasma polymer coating is formed on surfaces exposed to the plasma. This coating contains carbon in a non-crystalline form together with other elements from the carbon containing molecular species or other species co-released into the plasma. The surface may be heated or biased electrically during deposition. Such materials often contain unsatisfied bonds due to their amorphous nature.

In the case of adopting plasma treatment under plasma immersion ion implantation (PIII) and/or co-deposition and/or plasma polymer surface deposition conditions the present inventors have determined that not only is the substrate surface activated to allow binding of one or more peptides, but that the possibly hydrophobic nature of the surface is modified to exhibit a more hydrophilic character. This is important for maintaining the conformation and therefore functionality of many peptides/proteins, the outer regions of which are often hydrophilic in nature due to the generally aqueous environment of biological systems. Not only do techniques of the present invention give rise to hydrophilicity of the treated metal, semiconductor, polymer, composite and/or ceramic surfaces, but that as a result of cross linked sub-surface regions in the plasma polymer there is a delay to the hydrophobic recovery of the surface that takes place over time following the treatment, relative to polymer surfaces that are plasma treated but without energetic ion bombardment conditions. The inventors understand that the mechanism associated with delayed hydrophobic recovery is that in addition to the treatment giving rise to surface activation it also results in improved surface stabilisation. This stabilisation is understood to result from penetration into the sub-surface of the coating by energetic ions, giving rise to regions of cross-linking in the sub-surface. Although the surface is likely to be rough on an atomic scale, meaning that it is difficult to define the surface as a smooth plane, the energies of ions utilised will ensure that they penetrate at least about 0.5 nm into the interior of the deposited plasma polymer and up to about 500 nm from the growth surface during deposition. It is therefore intended for the term “sub-surface” to encompass a region, which may be the entire interior of the plasma polymer layer or the part of a polymer or plasma polymer, subject to energetic ion bombardment conditions, that is between about 0.5 nm and about 1000 nm beneath the final coating surface, preferably between about 3 nm and about 500 nm, 300 nm or 200 nm, and most preferably between about 5 nm and about 100 nm beneath the surface.

A plasma polymer surface can be generated through plasma ion implantation with carbon containing species, co-deposition under conditions in which substrate material is deposited with carbon containing species while gradually reducing substrate material proportion and increasing carbon containing species proportion and/or deposition of a plasma polymer surface layer with energetic ion bombardment. In this context the carbon containing species may comprise charged carbon atoms or other simple carbon containing molecules such as carbon dioxide, carbon monoxide, carbon tetrafluoride or optionally substituted branched or straight chain C₁ to C₁₂ alkane, alkene, alkyne or aryl compounds as well as compounds more conventionally thought of in polymer chemistry as monomer units for the generation of polymer compounds, such as n-hexane, allylamine, acetylene, ethylene, methane and ethanol. Additional suitable compounds may be drawn from the following non-exhaustive list: butane, propane, pentane, heptane, octane, cyclohexane, cycleoctane, dicyclopentadiene, cyclobutane, tetramethylaniline, methylcyclohexane and ethylcyclohexane, tricyclodecane, propene, allene, pentene, benzene, hexene, octene, cyclohexene, cycloheptene, butadiene, isobutylene, di-para-xylylene, propylene, methylcyclohexane, toluene, p-xylene, m-xylene, o-xylene, styrene, phenol, chlorphenol, chlorbenzene, fluorbenzene, bromphenol, ethylene glycol, diethlyene glycol, dimethyl ether, 2,4,6-trimethyl m-phenylenediamine, furan, thiophene, aniline, pyridine, benzylamine, pyrrole, propionitrole, acrylonitrile, pyrrolidine, butylamine, morpholine, tetrahydrofurane, dimethylformamide, dimethylsulfoxide, glycidyl methacrylate, acrylic acid, ethylene oxide, propylene oxide, ethanol, propanol, methanol, hexanol, acetone, formic acid, acetic acid, tetrafluormethane, fluorethylene, chloroform, tetrachlormethane, trichlormethane, trifluormethane, vinyliden chloride, vinyliden fluoride, hexamethyldisiloxane, triethylsiloxane, dioxane, perfluoro-octane, fluorocyclobutane, octafluorocyclobutane, vinyltriethoxysilane, octafluorotoluene, tetrafluoromethane, hexamethyldisiloxane, hepadecafluoro-1-decene, tetramethyldisilazane, decamethyl-cyclopentasiloxane, perfluoro(methylcyclohexane), 2-chloro-p-xylene.

The term “hydrophilic” refers to a surface that can be wetted by polar liquids such as water, and include surfaces having bath strongly and mildly hydrophilic wetting properties. For a smooth surface we use the term hydrophilic to mean a surface with water contact angles in the range from 0 to around 90 degrees. The most preferable water contact angle for the hydrophilic surfaces relating to the present invention are in the range of around 50 to about 70 degrees.

In one aspect a plasma polymer surface has a thickness of front about 0.3 nm to about 1000 nm, from about 3 nm to about 500 nm, 300 nm or 100 nm or from about 10 nm to about 30 nm. However, polymer surfaces on substrates or devices that are amenable to directionally orientated functionalisation with peptides according to the invention can be of any thickness—from a thin nanoscale coating, layer or plasma polymer deposit through to the situation wherein the substrate or device is a solid polymer material.

The terms “metal” or “metallic” as used herein to refer to elements, alloys or mixtures which exhibit or which exhibit at least in part metallic bonding. Preferred metals according to the invention include elemental iron, copper, zinc, lead, aluminium, titanium, gold, platinum, silver, cobalt, chromium, vanadium, tantalum, nickel, magnesium, manganese, molybdenum tungsten and alloys and mixtures thereof. Particularly preferred metal alloys according to the invention include cobalt chrome, nickel titanium, titanium vanadium aluminium and stainless steel.

The term “ceramic” as it is used herein is intended to encompass materials having a crystalline or at least partially crystalline structure formed essentially from inorganic and non-metallic compounds. They are generally formed from a molten mass that solidifies on cooling or are formed and either simultaneously or subsequently matured (sintered) by heating. Clay, glass, cement and porcelain products all fall within the category of ceramics and classes of ceramics include, for example, oxides, silicates, silicides, nitrides, carbides and phosphates. Particularly preferred ceramic compounds include magnesium oxide, aluminium oxide, hydroxyapatite, titanium nitride, titanium carbide, aluminium nitride, silicon oxide, zinc oxide and indium tin oxide.

The term “semiconductor” as it is used herein to refers to materials having higher resistivity than a conductor but lower resistivity than a resistor; that is, they demonstrate a band gap that can be usefully exploited in electrical and electronic applications such as in diodes, transistors, and integrated circuits. Examples of semiconductor materials include silicon, germanium, gallium arsenide, indium antimonide, diamond, amorphous carbon and amorphous silicon.

“Composite” materials comprehended by the present invention include those that are combinations or mixtures of other materials, such as composite metallic/ceramic materials (referred to as “cermets”) and composites of polymeric material including some metallic, ceramic or semiconductor content, components or elements. Such composites may comprise intimate mixtures of materials of different type or may comprises ordered, arrays or layers or defined elements of different materials.

The term “co-deposition” as used herein refers to a deposition process which deposits at least two species on a surface simultaneously, which may involve varying over time the proportions of the two or more components to achieve graded layers of surface deposition. Most preferably the deposition of this graded layer is commenced with deposition of only the substrate material, noting that layers deposited prior to the deposition of carbon containing species become the effective substrate.

By the term “mixed or graded interface” it is intended to denote a region in the material in which the relative proportions of two or more constituent components vary gradually according to a given profile. One method by which this mixed or graded interface is generated is by ion implantation. This achieves a transition from substrate material to deposited plasma polymer material. During the process any one of or any combination of the voltage, pulse length, frequency and duty cycle of the PIII pulses applied to the substrate may vary in time thereby varying the extent to which the species arising from the plasma are implanted. Another example method by which a graded metal/plasma polymer interface can be achieved is co-deposition, where the power supplied to the magnetron or cathodic arc source of metal, or the composition of the gases supplied to the process chamber are varied so that the deposited and/or implanted material changes progressively from more metallic to more polymeric.

The term “plasma” or “gas plasma” is used generally to describe the state of ionised vapour. A plasma consists of charged ions, molecules or molecular fragments (positive or negative), negatively charged electrons, and neutral species. As known in the art, a plasma may be generated by combustion, flames, physical shock, or preferably, by electrical discharge, such as a corona or glow discharge. In radiofrequency (RF) discharge, a substrate to be treated is placed in a vacuum chamber and vapour at low pressure is bled into the system. An electromagnetic field generated by a capacitive or inductive RF electrode is used to ionise the vapour. Free electrons in the vapour absorb energy from the electromagnetic field and ionise vapour molecules, in turn producing more electrons.

In conducting plasma treatment typically a plasma treatment apparatus (such as one incorporating a Helicon, parallel plate or hollow cathode plasma source or other inductively or capacitively coupled plasma source) is evacuated by attaching a vacuum nozzle to a vacuum pump. A suitable plasma forming vapour generated from a vapour, liquid or solid source is bled into the evacuated apparatus through a gas inlet until the desired vapour pressure in the chamber and differential across the chamber is obtained. An RF electromagnetic field is generated within the apparatus by applying current of the desired frequency to the electrodes from an RF generator. Ionisation of the vapour in the apparatus is induced by the electromagnetic field, and the resulting plasma modifies the metal, semiconductor, polymer, composite and/or ceramic substrate surface subjected to the treatment process.

In one embodiment of the invention it is possible to treat a plasma polymer surface either while it is being deposited or after its deposition, with a plasma forming vapour to thereby activate the plasma polymer surface for binding to peptides. Suitable plasma forming vapours used to treat the plasma polymer surface of the substrate include inorganic and/or organic gases/vapours. Inorganic gases are exemplified by helium, argon, nitrogen, neon, water vapour, nitrous oxide, nitrogen dioxide, oxygen, air, ammonia, carbon monoxide, carbon dioxide, hydrogen, chlorine, hydrogen chloride, bromine cyanide, sulfur dioxide, hydrogen sulfide, xenon, krypton, and the like. Organic gases are exemplified by methane, ethylene, n-hexane, benzene, formic acid, acetylene, pyridine, gases of organosilane, allylamine compounds and organopolysiloxane compounds, fluorocarbon and chlorofluorocarbon compounds and the like. In addition, the gas may be a vaporised organic material, such as an ethylenic monomer to be plasma polymerised or deposited on the surface. These gases may be used either singly or as a mixture of two more, according to need. Preferred plasma forming gases according to the present invention are argon, nitrogen and organic precursor vapours as well as inorganic vapours consisting of the same or similar species as found in the substrate.

Typical plasma treatment conditions (which are quoted here with reference to the power that may be required to treat a surface of 100 square centimetres, but which can be scaled according to the size of the system) may include power levels from about 1 watt to about 1000 watts, preferably between about 5 watts to about 500 watts, most preferably between about 30 watts to about 300 watts (an example of a suitable power is forward power of 100 watts and reverse power of 12 watts), frequency of about 1 kHz to 100 MHz, preferably about 15 kHz to about 50 MHz, more preferably from about 1 MHz to about 20 MHz (an example of a suitable frequency is about 13.5 MHz); axial plasma confining magnetic field strength of between about 0 G (that is, it is not essential for an axial magnetic field to be applied) to about 100 G, preferably between about 20 G to about 80 G, most preferably between about 40 G to about 60 G (an example of a suitable axial magnetic field strength is about 50 G); exposure times of about 5 seconds to 12 hours, preferably about 1 minute to 2 hours, more preferably between about 5 minutes and about 20 minutes (an example of a suitable exposure time is about 13 minutes); gas/vapour pressures of about 0.0001 to about 10 torr, preferably between about 0.0005 torr to about 0.1 torr, most preferably between about 0.001 torr and about 0.01 torr (an example of a suitable pressure is about 0.002 torr); and a gas flow rate of about 1 to about 2000 cm³/min.

According to the present invention deposition of a plasma polymer can be under plasma immersion ion implantation (PIII) conditions, with the intention of implanting the sub-surface of the substrate with energetic ions such as nitrogen, argon and/or organic carbon containing species to generate surface free radical activated sites. Typical PIII conditions include a substrate bias voltage to accelerate ions from the plasma into the treated substrate of between about 0.1 kV to about 150 kV, preferably between about 0.5 kV to about 100 kV, most preferably between about 1 kV to about 20 kV (an example of a suitable voltage is about 10 kV); frequency of between about 0.1 Hz to about 1 MHz, preferably between about 1 Hz to about 1000 Hz, most preferably between about 100 Hz to about 8000 Hz (an example of a suitable frequency is about 1000 Hz); pulse-length of between about 1 μs to about 1 ms, preferably between about 50 μs to about 500 μs (an example of a suitable pulse-length is about 50 μs). It is also possible to subject an existing polymer surface to energetic ion treatment to implant ionic species and generate radicals in the surface, without associated plasma deposition, using similar bias voltage conditions referred to above and non-depositing gases such as nitrogen and argon.

Following activation of the polymer substrate surface it is possible to functionalise the plasma polymer surface with a peptide by incubation (eg. by bathing, washing, stamping, printing or spraying the surface) of the activated polymer surface (substrate) with a solution comprising the peptide or a mixture of peptides, while manipulating the electronic field environment and/or charge of the surface and/or of the peptides to control orientation of peptide presentation and binding to the surface. Preferably the solution is an aqueous solution (eg. saline), that preferably includes a buffer system compatible with maintaining the biological function of the molecule, such as for example a phosphate or Tris buffer. It may then be appropriate to conduct one or more washing steps also using a biologically compatible solution or liquid, for example the same aqueous buffered solution as for the incubation (but which does not include the peptide), to remove any non-specifically bound material from the surface, before the functionalised polymer surface is ready to be put to its intended use. In another embodiment it is possible to use an agent such as bovine serum albumin (BSA) that will inhibit non-specific adsorption of further biological molecules.

By the phrase “manipulating the electronic field environment and/or charge” of the surface and/or of the peptides it is intended to convey that by controlling pH and/or salt concentration (ionic strength) of the incubation solution and/or by other means such as applying external electric potentials to the surface and/or solution it is possible to control electrostatic interactions between peptides and the polymer surface. For example, there may be oxygen species on the surface because many of the radicals react with environmental oxygen when they emerge at the surface and increasing the pH of the incubation solution above the pKa of these oxygen species on the polymer surface will make it energetically favourable for hydrogen atoms to be removed from oxygen species such as carboxyl groups or for cationic species to be removed from esters, with the result that these oxygen species are generally in a negatively charged state. Similarly, at increasing pH amino acids that are positively charged at neutral pH such as Arg and Lys will tend to lose a proton to adopt a neutrally charged state, whereas amino acids that are negatively charged at neutral pH will attract a proton at lower pH to adopt a neutrally charged state. Increasing ionic strength of the incubation solution will reduce the dimensions of double layers that screen charge on the surface and on the peptides and thereby increasing the field strength within these double layers.

The orientation of peptides approaching the surface during the incubation can also be controlled by the application of electric fields to the system. These can be applied via electrodes at the surfaces and/or in the incubation solution. In the case where a current cannot be conducted through the system due to insulators at the surfaces or interfaces, the electric field will be established in a double layer at the surface. On entering the double layer during their approach to the surface the peptides will feel the force of the electric field and their orientation will be influenced. If the incubation cell is able to conduct a current then an electric field would be established in the volume of the cell. This would be the case, for example, if a conducting polymer, such as polypyrrole, was used for immobilisation of the peptides. This electric field would provide an orienting force on the peptides as they approach the surface.

The orientation of binding of a particular peptide to a surface can be altered or switched by modifying incubation conditions, so that for example the peptide will bind in one orientation under one set of incubation conditions (e.g. at neutral pH) and with the other orientation under another set of incubation conditions (e.g. at acidic pH). Random or mixed orientation binding may be encouraged at an intermediate pH where there is no effective dipole moment in operation.

Patterning of substrates to provide defined regions with specifically oriented peptides can also be adopted. Shadow masks or contact masks applied during energetic ion treatment will result in the direct covalent binding capability of the surface to be restricted to only the areas exposed to treatment so that the masked areas will not covalently attach peptide. Any peptide physically adsorbed during incubation on the masked sites can be removed by a gentle detergent wash (e.g. Tween).

To achieve patterning with respect to the orientation of covalently immobilised peptide, the surface area over which a peptide incubation solution is applied can be limited. This can be done by using gaskets to limit the region over which each peptide containing solution can contact the surface. It is also possible to apply a limited volume of solution so that it will be restricted to a small area near the point of application. This process can be repeated with numerous solutions enabling the patterning of various peptides in a range of orientations as determined by the solution conditions used in each incubation step.

In other broad aspects of the invention there is provided a polymer substrate surface having a peptide directly covalently bound thereto in a manner wherein the predominant orientation of binding of the peptide to the surface is controlled that is produced by a method as outlined above, as well as devices that comprise such surfaces. The invention also generally provides polymer substrate surfaces having a peptide directly covalently bound thereto in a manner wherein the predominant orientation of binding of the peptide to the surface is controlled, and devices comprising such surfaces.

The inventors have determined that substrates functionalised with peptides according to the invention exhibit extensive shelf life. For example, the functionalised substrates can be stored (ideally in a sealed environment, following freeze drying or in a sealed environment at low temperature)) for a period of minutes, hours, days, weeks months or years without significant degradation before being re-hydrated, if necessary, and put to their intended use. If freeze drying is adopted a stabiliser such as sucrose may beneficially be added before the freeze drying process. The sealed environment is preferably in the presence of a desiccant and may comprise a container or vessel (preferably under vacuum or reduced oxygen atmosphere) or may for example comprise a polymer, foil and/or laminate package that is preferably vacuum packed. Preferably the sealed environment is sterile to thus prevent or at least minimise the presence of agents such as proteases and nucleases that may be detrimental to activity of the biological molecules. Alternatively the functionalised substrates and devices may be stored in a conventional buffer solution, such as mentioned above, as appropriate depending upon the nature of the substrate or device.

The invention will now be described further, and by way of example only, with reference to the following non-limiting examples.

### EXAMPLES

### Materials

PFTE and polystyrene were obtained from Goodfellow. PEEK was sourced from Vitrex. Peptide 36 (ACLGKACGRKRK) and peptide 36 short (ACLGKACG) were synthesized by Auspep. Recombinant human tropoelastin corresponding to amino acid residues 27-724 of GenBank entry AAC98394 (gi 182020) was expressed and purified as previously described [1]. Human dermal fibroblasts were cultured in a humidified 5% CO₂ atmosphere in DMEM (Invitrogen) supplemented with 10% (v/v) fetal calf serum (Invitrogen), and passaged 1 in 10 every 3-4 days. Unless stated otherwise all other reagents were purchased from Sigma.

### Methods

### Surface Treatment

Polymeric sheets were mounted onto a substrate holder and a metallic mesh, electrically connected to the substrate holder, was mounted 5.5 cm in front of the polymer surface. For plasma treatment the substrate holder and mesh were immersed in an inductively coupled 1000 W rf plasma for increasing durations. The working gas pressure was 2 mTorr with a flow rate of 72 standard cubic cm of high purity nitrogen (99.999%). Where applicable the samples were PIII treated by applying 20 kV pulses each lasting for 20 μs with a repetition rate of 50 Hz to the sample holder during plasma treatment. The sample holder was earthed between pulses. More details of the treatment parameters and resulting surface properties can be found in [1].

Samples were contact or shadow masked during PIII treatment by layering 3 mm wide low contact ADH kapton tape (contact mask) (Associated Gaskets, Australia) or a 130 μm stainless steel plate (shadow mask) (Mastercut Technologies, Australia) over the sample during PIII treatment. The masks were removed immediately after treatment.

In cases where the samples were allylamine treated, they were placed in contact with allylamine immediately after PIII treatment. The samples were removed from the PIII chamber and rapidly placed on a wire gauze suspended in a glass vessel above allylamine solution. The air was displaced with argon and the vessel sealed. After the samples Were subjected to increasing durations of incubation in allylamine vapors they were transferred to a clean glass vessel, the air displaced with argon and the vessel sealed. Orange II dye and cell analysis were carried out within 2 hours of allylamine vapor treatment to reduce the reaction of oxygen with the surfaces.

### ATR FTIR

Surface-bound peptide was detected using ATR-FTIR as previously described [2]. The ATR-FTIR spectra were recorded using a Digilab FTS7000 FTIR spectrometer fitted with an ATR accessory with a trapezium germanium crystal employing an incidence angle of 45°. 500 scans with a resolution of 1 cm⁻¹ were taken to enable spectra with sufficiently high spectral band resolution and signal to noise ratio to be generated.

The samples were immersed in 50 μM peptide 36 solution for 1 hour. Non-bound peptide was removed with extensive PBS washing then the salts were removed by extensive milliQ H₂O washing followed by air drying for 3 days prior to FTIR measurement. Selected samples were washed also with 2% tween-20 at 70° C. for 20 min, washed extensively with PBS, then milliQ H₂O and air dried for 3 days before their ATR-FTIR spectra were taken.

All spectra are shown as difference spectra, obtained by subtracting the spectra of control surfaces that were identically processed except that the peptide solution incubation step was performed in PBS only. GRAMS software was used for spectral analysis.

### Cell Attachment

Samples were coated with peptide 36 diluted to the appropriate concentration in 10 mM PO₄ buffer containing varying NaCl concentrations and pH. The samples were incubated in peptide 36 for 1 hour at room temperature then unbound peptide 36 was removed with 3×PBS washes. Where stated samples were incubated in 2% Tween-20 for 10 min at 70° C. then washed with PBS. Where stated non-specific cell binding was prevented with incubation in 10 mg/ml heat denatured (80° C./10 min) BSA for 1 hour at room temperature. Confluent 75 cm² flasks of cells were harvested by trypsinization, and the cell density adjusted to 5×10⁵ cells/ml in serum free DMEM. The cells were added to the samples for 60 min at 37° C., 5% CO₂ then non-adherent cells were removed with 2×1 PBS washes. Adherent cells were fixed with the addition of 5% glutaraldehyde (w/v) in PBS for 20 min. The samples were washed 3×PBS, and then the cells were stained with 0.1% (w/v) crystal violet in 0.2M MES pH 5.0 for 1 h at room temperature. After washing extensively with dH₂O the samples were transferred to fresh tissue culture plates and the crystal violet was solubilised in 10% (v/v) acetic acid. The absorbance was measured at 570 nm using a plate reader. The absorbance was converted to % cell attachment using the equation of a linear regression fit of standard known cell number controls.

### Cell Morphology

Samples were peptide 36 coated, BSA blocked and cell seeded as for cell attachment. After incubation for 90 min the cells were fixed with the addition of 37% (w/v) formaldehyde directly to the cell media to achieve a final concentration of 3% (w/v) for 20 min. For opaque samples the cells were crystal violet stained as for cell attachment. The cell morphology was then observed by phase contrast microscopy.

### Toluidine Blue Detection of Surface COOH Groups

Surface negatively charged groups were detected as in [3]. 1.2×0.8 cm samples were incubated in 2 mM toluidine-blue-O, 15 mM NaCl, pH 11 for 10 min at room temperature. Non-bound toluidine blue was removed with 4×2 hour washes in 15 mM NaCl pH 11 with agitation. Bound toluidine blue was then eluted in 200 mM NaCl, pH 2 for 30 min and the absorbance measured at 630 nm. Samples not exposed to toluidine blue were used as background controls.

### Orange II Dye Detection of Surface Amine Groups

A calorimetric assay was employed to quantify C═NH/C—NH₂ groups on the surfaces [4]. The samples were incubated in 15 mg/ml orange II dye, pH 3 for 40 min at 37° C. Unbound dye was removed with 3×washes of pH 3 water. The samples were air dried for 1 hour at room temperature then the bound dye was eluted with pH 12 water for 20 min with agitation. The solution was adjusted to pH 3 with HCl and the absorbance read at 490 nm.

### C-Terminal Antibody

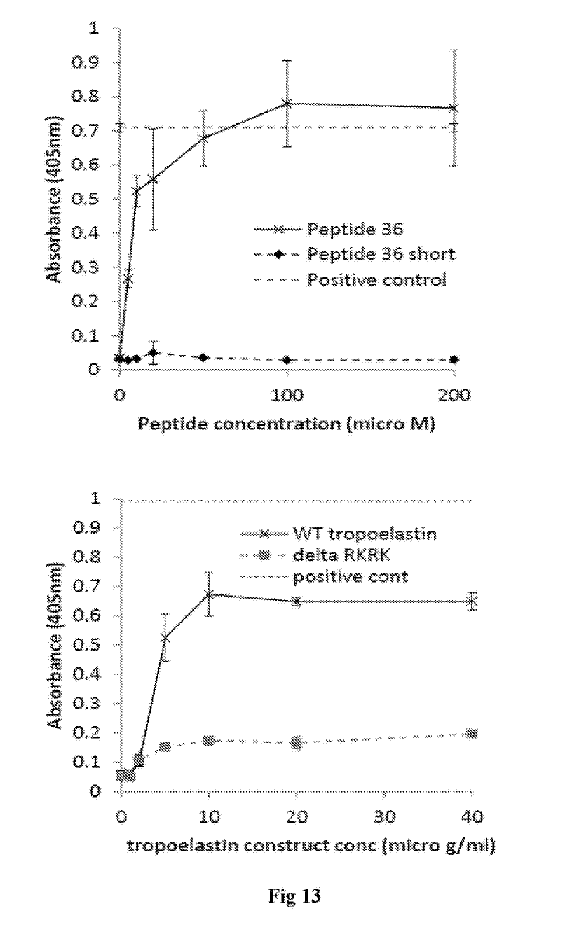

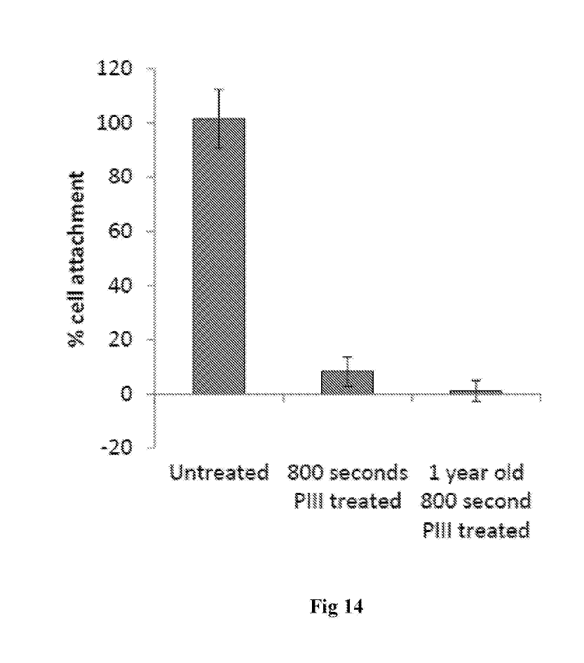

Rabbit polyclonal antibodies against exon 36 were raised by Biomatik. A peptide corresponding to exon 36 (GGACLGKACGRKRK) conjugated to Keyhole Limpet Hemocyanin was used to immunize New Zealand rabbits. Exon 36 specific antibodies were purified from the resulting immune serum by affinity chromatography using the immunogen peptide. The resulting antibody showed a high degree of specificity against the RKRK motif within exon 36 as by ELISA it detected peptide 36 but not ΔRKRK peptide 36 where the RKRK motif was deleted (FIG. 13A). This was confirmed by increased ELISA detection of recombinant tropoelastin versus ΔRKRK tropoelastin in which the C-terminal RKRK motif had been deleted (FIG. 13B). Therefore this protocol produced a rabbit polyclonal antibody with specificity against the C-terminal RKRK motif of tropoelastin.

### C-Terminal ELISA

Untreated or PIII treated polystyrene was incubated in peptide 36 containing buffer (10 mM PO₄, 150 mM NaCl) of increasing pH for 1 hour at room temperature. After peptide immobilization the samples were washed 3× with PBS to remove non-bound peptide. Background antibody binding was blocked with 3% (w/v) BSA for 1 hour at room temperature. The primary rabbit-anti-C-terminal antibody was diluted to 1:5000 and added to the samples for 1 hour at room temperature. Non-bound antibody was removed with 3×PBS washes then detected with 1:10,000 diluted goat anti-rabbit whole IgG-HRP conjugated secondary antibody for 1 hour at room temperature. The secondary antibody was removed, the samples washed 3×PBS washes and then transferred to fresh tissue culture plates. ABTS solution (40 mM ABTS, 0.1 mM NaOAc, 0.05M NaH₂PO₄, 0.01% H₂O₂, pH 5) was added for 30 min and the absorbance read at 405 nm.

### Autoclaving

Samples were peptide coated as for cell attachment analysis then washed 3× with PBS. Where stated the samples were placed in a dry beaker (dry autoclave) or in 10 ml of PBS (liquid autoclave). The samples were then autoclaved at 120° C., 100 kPa above atmospheric pressure with steam for 20 min. For slow cooled samples the temperature of the autoclave returned to room temperature over a period of 4 hours. The other samples were removed once the autoclave reached 70° C. and were immediately immersed in room temperature PBS. All samples were washed 3× with PBS prior to measuring cell attachment.

### Example 1

### Peptide 36 Interactions with PIII Treated Polystyrene

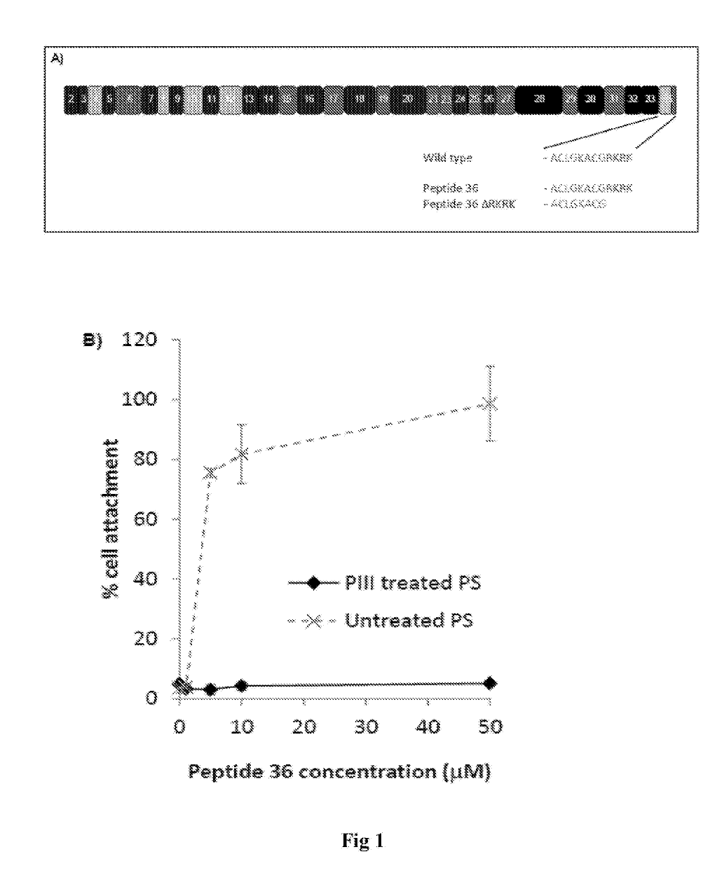

We have previously detailed the surface induced modulation of tropoelastin-cell binding by PIII treatment of PTFE [5, 6]. This activity modulation was attributed to differential exposure of the C-terminus of tropoelastin. Previously using a biochemical approach we have identified peptide 36 (ACLGKACGRKRK) from exon 36 (FIG. 1A) as a major cell binding motif that is present at the C-terminus of tropoelastin [7]. We therefore tested if peptide 36, in isolation from the remainder of the tropoelastin molecule could support cell binding on untreated and PIII treated polystyrene (FIG. 1B). When coated onto PIII treated polystyrene peptide 36 did not support cell binding above BSA background controls with maximal 5.1±0.5% cell attachment using a coating concentration of 50 μM. In contrast peptide 36 supported cell attachment in a dose dependent manner on untreated polystyrene. Maximal 98.6±12.5% peptide 36-dependent cell attachment was observed with a coating concentration of 50 μM. The maximal cell attachment to peptide 36 on untreated polystyrene is similar to tropoelastin on untreated PTFE [5]. This shows that peptide 36 can recapitulate the full cell-binding activity of intact tropoelastin and that this activity is modulated by the underlying polymer treatment.

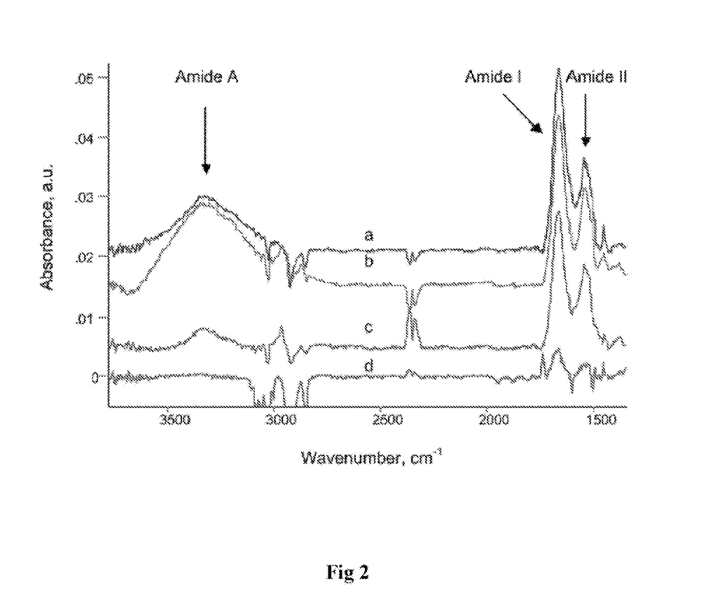

Proteins, including tropoelastin bind covalently to PIII treated polymers through a radical dependent mechanism [8]. We therefore sought to determine if peptide 36 can bind covalently to PIII treated polystyrene. To detect covalent tropoelastin binding an ELISA utilizing the tropoelastin-specific BA-4 antibody was used. Peptide 36 does not contain the antibody epitope for BA-4, and so we used ATR-FTIR to identify peptide-associated amide bonds on the coated surfaces (FIG. 2). Amide bands corresponding to Amide A (˜3300 nm), Amide I (˜1650 nm) and Amide II (˜1550 nm) were observed on peptide 36 coated untreated and PIII treated polystyrene. After tween-20 washing amide bands were still observed on PIII treated polystyrene but not on untreated polystyrene. Tween-20 was used as a washing detergent in preference to SDS because it does not contain sulfur. This allowed XPS detection of peptide associated sulfur which correlated with the ATR-FTIR data (data not shown). Therefore consistent with previously observed covalent protein binding, peptide 36 binds covalently to PIII treated polymers.

### Example 2

### Surface Modulation of RKRK Dependent Cell Binding to Peptide 36

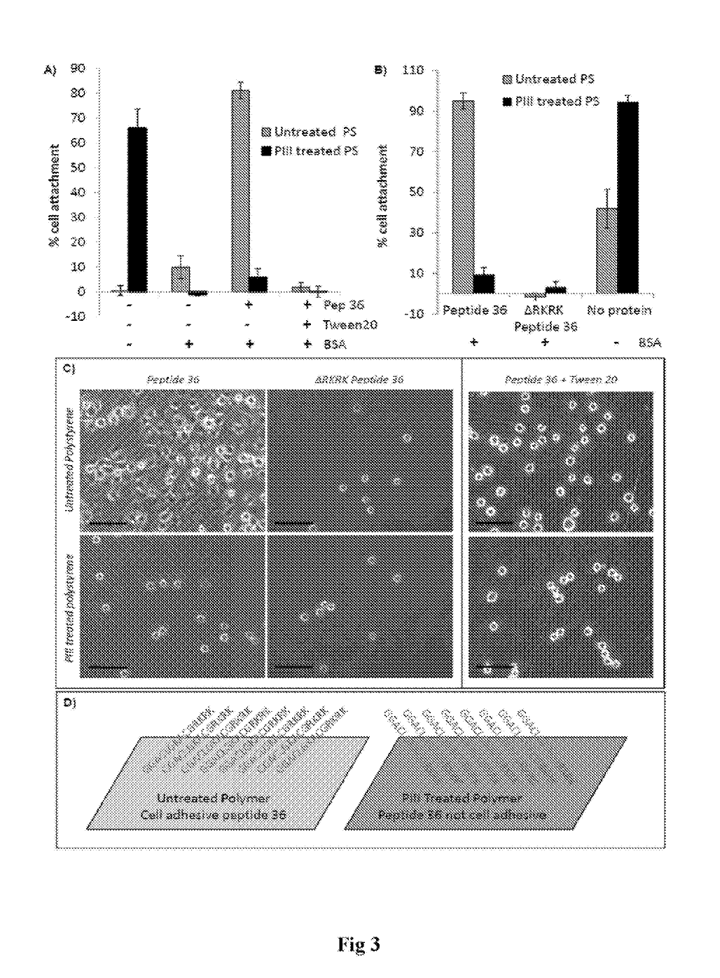

Cell binding assays were used to further probe the cell binding activity of peptide 36 on untreated versus PIII treated polystyrene (FIG. 3). Although PIII treated polystyrene supports high levels of cell attachment in the absence of protein coating, peptide 36 is not cell adhesive on this surface (FIG. 3A). In contrast untreated polystyrene possesses low levels of cell binding in the absence of protein coating. However upon peptide 36 coating untreated polystyrene supports high levels of cell adhesion. Consistent with peptide 36 removal from untreated polystyrene, tween-20 washing reduced peptide 36 dependent binding on untreated polystyrene to background levels. Furthermore ΔRKRK peptide 36 did not support cell binding on either surface (FIG. 3B). Therefore the C-terminal RKRK motif is critical for peptide 36-cell binding on these surfaces. Peptide 36-dependent cell spreading showed a similar profile where cells became phase dark and flattened on peptide 36 coated untreated polystyrene only (FIG. 3C). Tween-20 washing removed peptide 36 dependent cell spreading activity from the untreated polymer. The ΔRKRK (peptide 36 did not elicit cell spreading on either the untreated or the PIII treated polystyrene surface. Therefore peptide 36 dependent cell attachment and spreading require the C-terminal RKRK motif and are modulated by the underlying material (FIG. 3D).

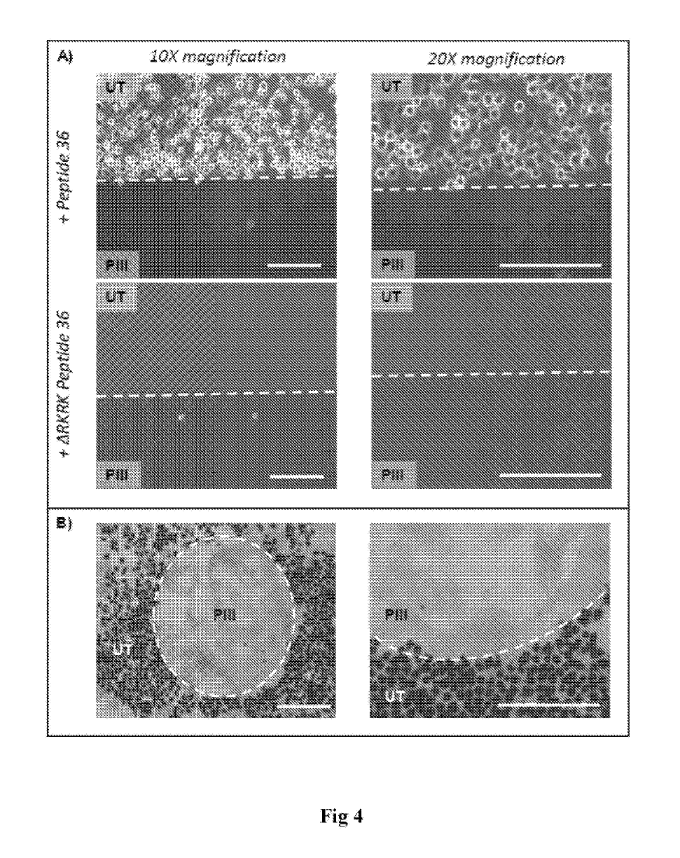

Contact (tape) masks and shadow masks are frequently used to limit PIII treatment to specific areas of a polymer surface [6]. Therefore as a method to achieve a patterned cell distribution on peptide 36 coated polystyrene we used patterned PIII treatment in conjunction with peptide 36 coating (FIG. 4). Using either a tape or shadow masking approach peptide 36-dependent cell attachment was clearly restricted to the untreated regions of the polymer surface. Cells attached and spread up to the boundary between the untreated and PIII treated regions but would not cross the boundary into the PIII treated region. The importance of the C-terminal RKRK motif was illustrated by the absence of cell attachment and spreading on the ΔRKRK peptide 36 coated material.

### Example 3

### Surface Charge Modulation of Peptide 36-Directed Cell Attachment

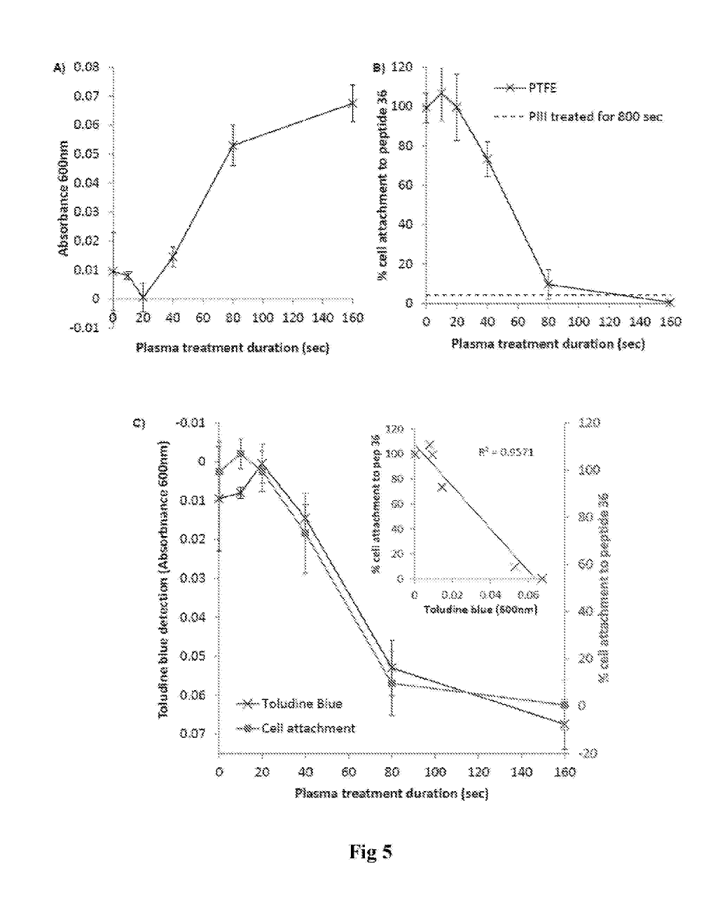

PIII treatment breaks bonds in polymers resulting in the formation of high energy radicals. When exposed to air these radicals can react with atmospheric oxygen, forming oxidized chemical groups such as ester, carbonyl and carboxyl groups. This results in increased polarity of the surface with a net negative charge [9, 10]. Peptide 36 has a strongly positively charged region encompassed by the RKRK C-terminal cell binding site. Therefore we proposed that electrostatic interactions with the negatively charged PIII treated surface are orientating peptide 36, thereby sterically hindering cell engagement with the peptide (FIG. 3D). To determine if such negatively charged COOH groups could be responsible for peptide 36 activity modulation we measured surface COOH groups using toluidine blue O on plasma treated PTFE (FIG. 5A). Plasma was used in preference to PIII treatment so that a gradual increase in fluence could be achieved with increasing treatment duration. In the absence of plasma treatment untreated PTFE had low levels of surface COOH groups. This remained unchanged using plasma treatment durations of up to 20 seconds. Plasma treatments above 20 seconds resulted in a dose dependent increase in toluidine blue O detection which approached a plateau with 160 seconds of plasma treatment. It should be noted that the samples were exposed to air for 14 days post plasma treatment which has been previously shown to allow saturation of the oxygen group formation on treated PTFE surfaces [1]. Alongside toluidine blue O staining we measured peptide 36 dependent-cell binding on these plasma treated PTFE surfaces (FIG. 5B). With plasma treatments up to 20 seconds PTFE surfaces could support high levels of peptide 36 dependent cell binding (99.3-106.9% cell attachment). With plasma treatments of longer than 20 seconds there was a plasma-dose dependent reduction in peptide 36 dependent cell attachment until background levels of peptide 36-cell attachment were observed with 160 seconds of plasma treatment. This correlated well to the peptide 36-cell attachment of 4.3±1.8% which was observed on 800 second PIII treated PTFE. Superimposed, the toluidine blue O detection of COOH groups and cell attachment to peptide 36 data showed a strong (R²=0.957) linear relationship between suit ace COOH groups and peptide 36-cell binding activity (FIG. 5C).

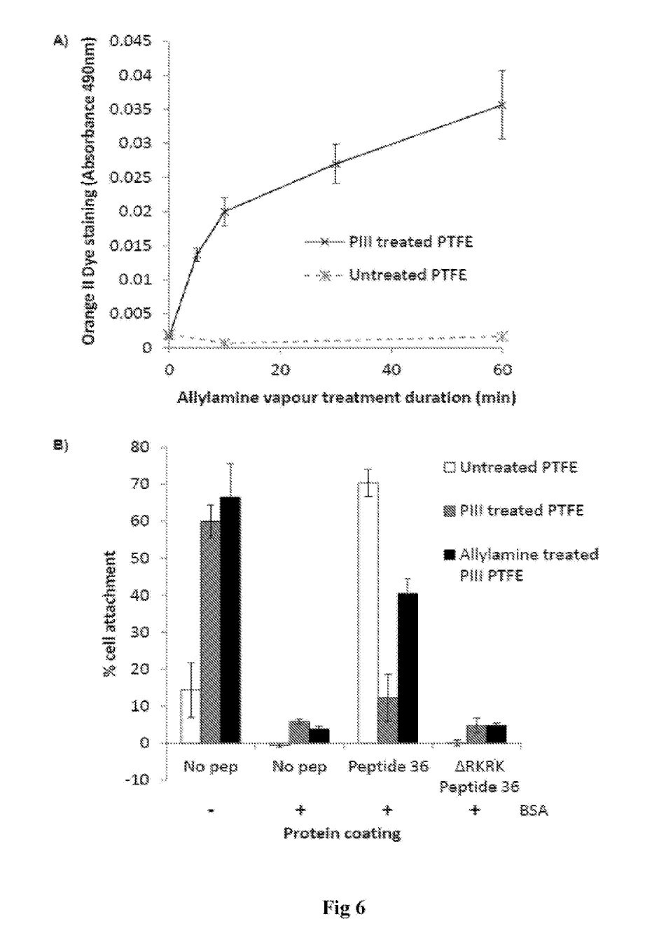

To further analyze the effect of surface charge on peptide 36-cell binding activity we modified the charge on the PIII treated surfaces using allylamine. Allylamine is a highly aminated compound which contains a C═C bond that can react with free radicals, thereby binding amine groups to the surface. These impart a positive charge to the surface [9]. When untreated PTFE films were exposed to allylamine vapor treatment for increasing durations no surface amine groups were detected using orange II dye (FIG. 6A). By contrast on PIII treated PTFE a dose dependent increase in surface amine groups was observed with increasing durations of allylamine vapor exposure. In the absence of protein, allylamine vapor treated and PIII treated PTFE both supported high levels of cell attachment whereas untreated PTFE supported low levels of cell attachment. This indicates that allylamine treatment is non-toxic to cells, at least over the durations of cell attachment analysis. Peptide 36 showed high levels of cell binding on untreated PTFE and low levels on PIII treated PTFE. Consistent with our hypothesis of a charged based mechanism for modulation of peptide 36-directed cell binding activity, peptide 36 showed far greater levels of cell binding activity on the post PIII allylamine treated PTFE than on PIII treated PTFE. Peptide 36-directed cell binding activity was lower on post PIII allylamine treated PTFE than untreated PTFE. This may be because it is very difficult to completely exclude oxygen from the surfaces. Therefore surface COOH groups may have formed in addition to the amine groups associated with the immobilized allylamine. This could then result in a heterogeneous surface with positively charged amine and negatively charged carboxylic acid entities and so heterogeneous peptide 36 modulation on this surface. The ΔRKRK peptide 36 showed low levels of cell binding activity on all of the surfaces tested, reinforcing the requirement for the RKRK for peptide 36-cell binding activity.

Taken together these data highlight the importance of surface charge for surface-induced modulation of peptide 36-cell binding activity.

### Example 4

### Peptide Charging and Ionic Buffer Conditions Influence Surface-Induced Peptide 36-Directed Cell Binding Activity Modulation

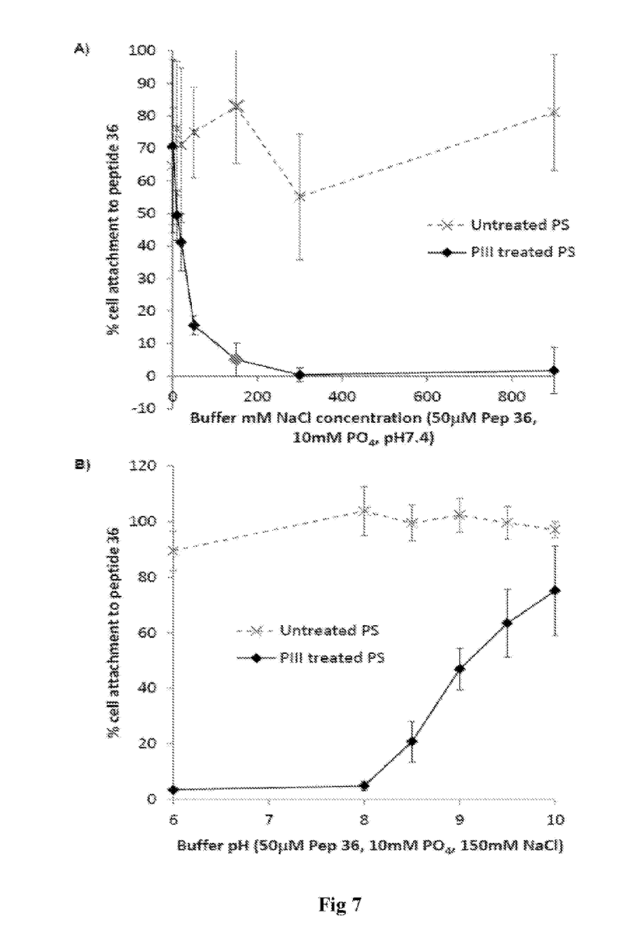

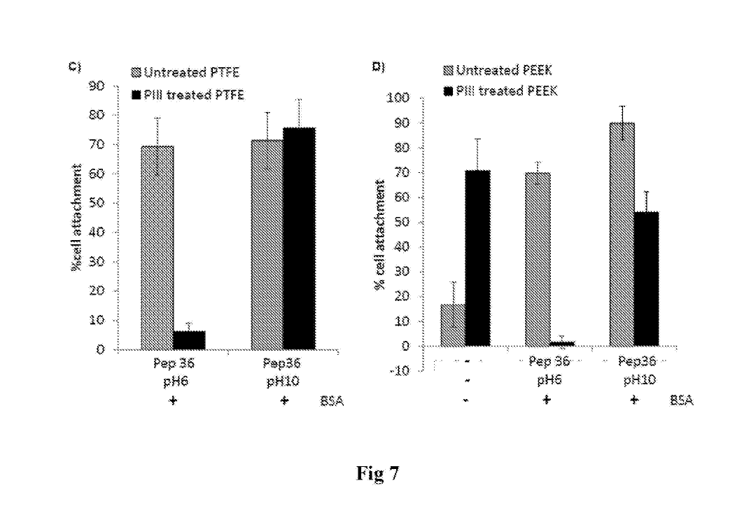

Peptide 36-surface electrostatic interactions are important for peptide 36-directed cell binding activity. Therefore to influence potential charge interactions increasing NaCl conditions were employed during peptide 36-surface association (FIG. 7A). On untreated polystyrene peptide 36 possessed high levels of cell-binding activity, independent of the NaCl content in the association buffer. In contrast NaCl heavily influenced peptide 36-cell binding activity on treated PTFE. Using a low ionic strength buffer during peptide 36-surface association resulted in high levels of peptide 36-cell binding activity. With increasing NaCl content the cell binding activity of bound peptide 36 reduced in a NaCl dose dependent manner until background levels of binding were observed with 150 mM NaCl. It should be noted that the NaCl content was only altered during the peptide 36-surface association Surfaces were returned to 150 mM NaCl containing buffer for BSA blocking and cell attachment. Additionally peptide 36 has been observed by ATR-FTIR on both untreated and PIII treated polystyrene using a 150 mM NaCl containing buffer (FIG. 2). Therefore the lack of cell binding activity with increasing NaCl buffer content is unlikely to be due to a lack of peptide 36 binding to PIII treated polystyrene. As such we have shown that NaCl content can influence peptide 36 activity in a manner that is resistant to subsequent changes in buffer ionic strength.

As a method to ascertain the importance of peptide charging we incubated untreated and PIII treated polystyrene with peptide 36 in buffers of increasing pH (FIG. 7B) With increasing pH the PIII treated polystyrene surface COOH groups should remain as COO⁻ entities as the pKa for COOH groups is approximately 2. Conversely with increasing pH the amine groups on the Arg and in particular the Lys side chains of peptide 36 should become neutral (NH₃⁺ to NH₂). On untreated polystyrene peptide 36 possesses high levels of cell binding activity. This was insensitive to the pH of the association buffer. In contrast on PIII treated polystyrene peptide 36 possessed low levels of cell biding activity with association buffers of up to pH 8. With buffers of pH 8 and above there was a pH dependent increase in the cell binding activity of surface bound peptide 36 with maximal cell binding activity at pH 10. Increasing the pH of the association buffer above 10 did not significantly alter the cell binding activity of peptide 36 (data not shown). This pH effect was independent of the polymeric surface utilized (FIGS. 7C, D). On both PIII treated PTFE and PEEK cells bound to peptide 36 when coated to the untreated polymer. When coated onto PIII treated polymer peptide 36 exhibited low levels of cell binding activity from a pH 6 association buffer. Conversely peptide 36 possessed high levels of cell binding activity when coated onto PIII treated polymer from a pH 10 buffer. Therefore charge dependent modulation of peptide 36 cell binding activity is not polymer specific but instead is general to the polymers tested. Again the pH was only altered dining the peptide 36-surface association, and so association buffer pH can influence peptide 36 activity in a manner that is resistant to subsequent changes in buffer pH. It is unlikely that the low levels of cell binding to peptide 36 coated PIII treated polystyrene at pH 8 and below is due to a lack of peptide 36 binding as ATR-FTIR identified association with PIII treated polystyrene from a similar pH 7.4 butter (FIG. 2)

### Example 5

### Peptide 36 Cell Binding Activity is Dependent upon RKRK Solvent Exposure

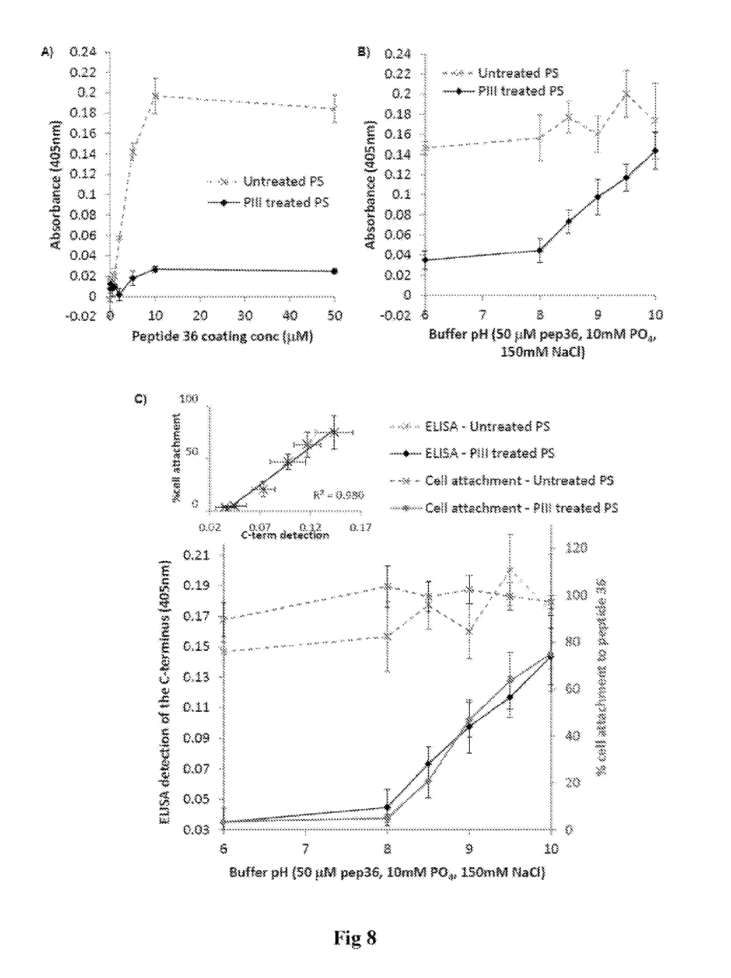

To enable detection of peptide 36 on our surfaces we generated a rabbit polyclonal antibody against exon 36 of tropoelastin. The resulting antibody was specific to the presence of the C-terminal RKRK motif of peptide 36 as it was not capable of detecting ΔRKRK peptide 36 or ΔRKRK tropoelastin by ELISA (FIG. 13). This antibody specificity for the C-terminal RKRK motif allowed us to measure peptide 36-RKRK exposure on untreated and PIII treated polystyrene (FIG. 8A). On untreated polystyrene the RKRK-sensitive antibody detected peptide 36 bound to the surface in a coating concentration dependent manner. Increasing coating resulted in increased ELISA detection up to a peptide 36 coating concentration of 10 μM. No increases in detection were noted with a coating concentration of 50 μM. Conversely little RKRK-dependent antibody detection was observed on PIII treated polystyrene with coating concentrations up to 50 μM. Therefore there is differential display of the RKRK motif of peptide 36 on untreated versus PIII treated polystyrene when association occurs in a 10 mM PO₄, 150 mM NaCl, pH 7.4 peptide buffer.

Previously we have noted that the cell binding activity of peptide 36 coated on to PIII treated polystyrene is sensitive to the pH of the association buffer. To determine if this is due to differential display of the C-terminal RKRK motif we ascertained the level of RKRK exposure using the RKRK-sensitive antibody (FIG. 8B). On untreated polystyrene the RKRK-sensitive antibody detected peptide 36 across the entire range of pH of association buffers tested. In agreement with RKRK requirement for cell-binding, the RKRK-sensitive antibody detected low levels of peptide 36 RKRK exposure on PIII treated polystyrene with association buffers with pH up to 8 Using association buffers with pH above 8 there was a pH dependent increase of RKRK detection with increasing buffer pH. Indeed, using a pH 10 peptide 36 association buffer the levels of RKRK detection were not statistically different to those obtained on untreated polystyrene. When the pH dependent RKRK detection data was overlaid with the pH dependent peptide 36-cell binding data (from FIG. 7B) there was a correlation of peptide 36 dependent cell-binding activity and RKRK detection using the RKRK sensitive antibody (FIG. 8C). This correlation showed a linear relationship between RKRK detection and cell binding (R²=0.98).

Together these data indicate that surface charge, peptide charge and buffer ionic strength modulate the cell binding properties of peptide 36 bound to PIII treated polymers by influencing the exposure of the cell-binding C-terminal RKRK motif.

### Example 6

### Temperature Passivation of Peptide 36 Cell Binding Activity

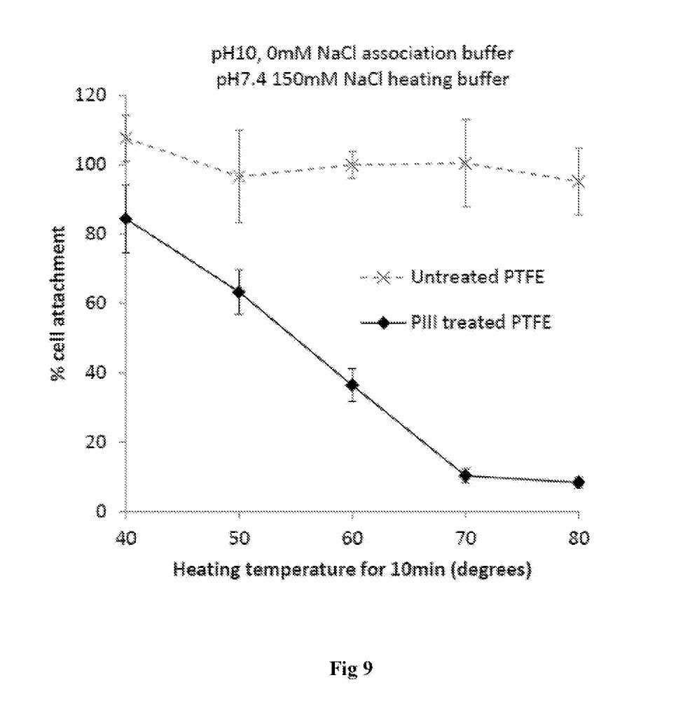

Protein/peptide motion increases with heating. To delineate if peptide motion can influence peptide 36-directed cell binding activity, peptide coated untreated and PIII treated surfaces were heated. The peptide was coated onto the surfaces in 10 mM PO₄, 0 mM NaCl pH 10 buffer known to allow cell binding activity of peptide 36 on both untreated and PIII treated PTFE (FIG. 9). Heating was conducted in 10 mM PO₄, 150 mM NaCl pH 7.4 buffer. With incubation temperatures up to 40° C., peptide 36 remained cell adhesive on both the untreated and PIII treated PTFE surfaces. When the incubation temperature was increased above 40° C. the cell binding activity of peptide 36 was not altered on untreated PTFE. In contrast on PIII treated PTFE the cell-binding activity of bound peptide 36 decreased with increasing incubation temperature to baseline levels of cell binding with incubations of 70° C. and above.

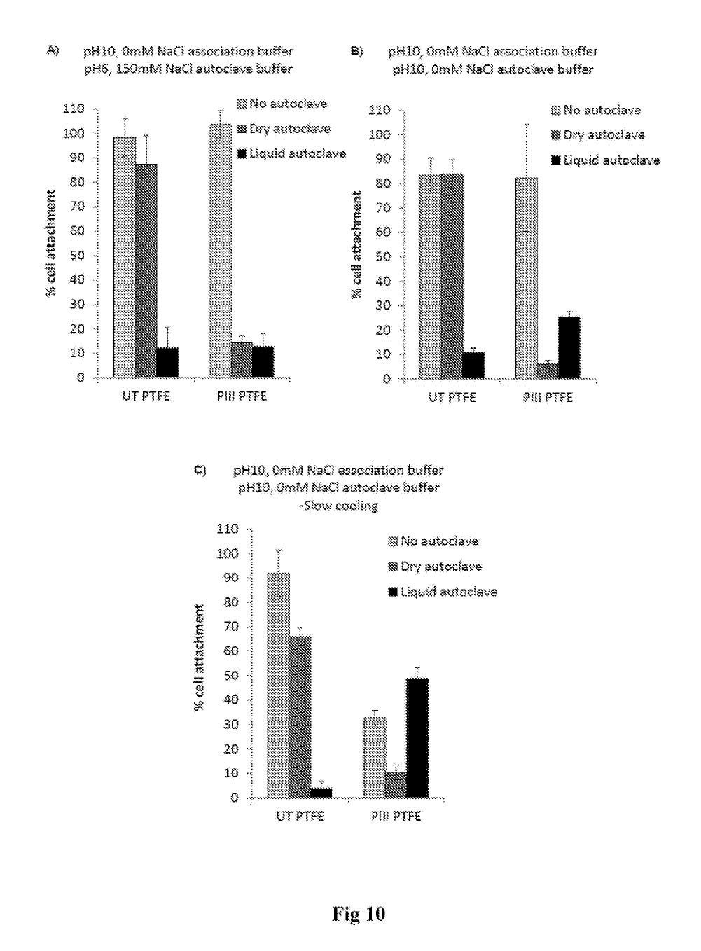

For the utilization of peptide coated surfaces in tissue engineering applications it is beneficial if the surface is amenable to sterilization post peptide-coating. To determine if peptide 36 coated surfaces were amenable to such sterilization they were subjected to autoclaving under dry or liquid conditions (FIG. 10). Consistent with previous data, peptide 36 was cell adhesive on both untreated and PIII treated PTFE when coated in pH 10, 0 mM NaCl buffer. Peptide 36-directed cell binding activity was retained on untreated PTFE after dry autoclaving. This suggests that autoclaving does not affect the cell binding properties of peptide 36. On PIII treated PTFE peptide 36 is not cell adhesive after dry autoclaving. This is consistent with the temperature dependent (80° C.) reduction in peptide 36-cell binding activity observed earlier on PIII treated PTFE (FIG. 9). When autoclaved in liquid conditions peptide 36-cell binding activity was lost on untreated PTFE regardless of the liquid buffer conditions employed. When using a pH 6, 150 mM NaCl liquid autoclave procedure the cell binding activity of peptide 36 was reduced to background levels on PIII treated PTFE. Instead partial peptide 36-cell binding activity was retained on PIII treated PTFE if pH 10, 0 mM NaCl buffer was used for liquid autoclaving. To retain full peptide 36-cell binding activity on PIII treated PTFE a pH 10, 0 mM NaCl liquid buffer was used, however a gradual cooling to room temperature procedure was implemented instead of rapid cooling. Under these conditions PIII treated PTFE retained comparable peptide 36-cell binding activity to the no-autoclave control. Therefore by utilizing appropriate autoclaving conditions it is possible to sterilise PIII treated PTFE surfaces post peptide 36 coating and retain peptide 36 activity.

### Example 7

### Patterned Cell Distribution on Peptide 36 Coated Polymers

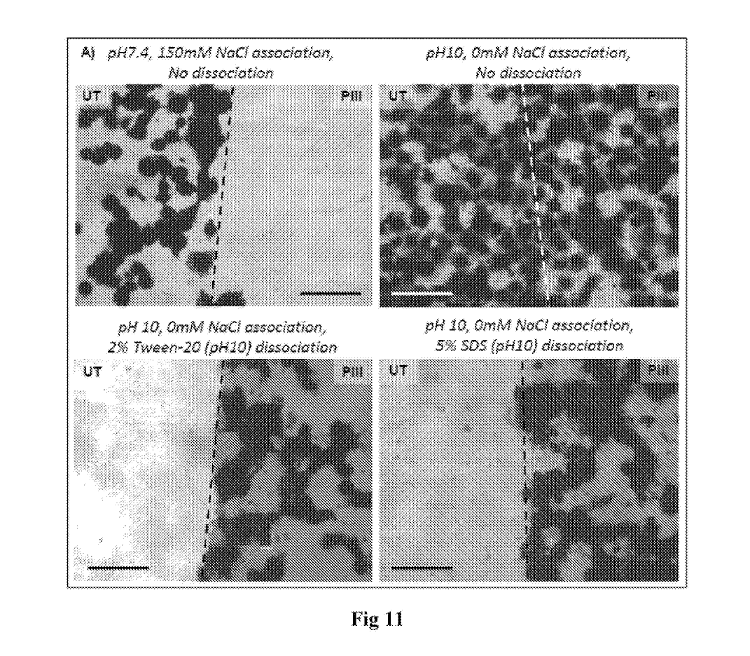

Controlled cell patterning is a requirement for a wide range of cell-based technologies such as fabrication of cell-based biosensors, cell separation techniques and for the ability to regenerate tissue architecture on biomaterials. Using the electrostatic orientation of peptide 36 and covalent peptide binding characteristics of PIII treated polymer it is possible to generate fine cell distributions (FIG. 11). Using an appropriate association buffer (pH 7.4, 150 mM NaCl) cells are retained selectively by peptide 36 on the untreated regions of a tape masked PIII treated PTFE surface. Few cells are observed on the PIII treated regions. This patterning is due to the electrostatic based modulation of peptide 36-cell binding activity. It is unlikely to be due to differential peptide 36 coating to the surface as ATR-FTIR showed peptide associated amide bonds on both untreated and PIII treated polymer under such association conditions. Using different association buffer conditions (pH 10, 0 mM NaCl) it is possible to achieve peptide 36-dependent cell binding on both the untreated and PIII treated sections. This is due to the abolition of electrostatic orientation of peptide 36 on PIII treated polymers. By contrast where peptide 36-cell attachment is desired exclusively on the PIII treated areas with covalently immobilised peptide, the surface is washed using 2% Tween-20 or 5% SDS in pH 10, 0 mM NaCl containing buffer. Using this buffer to wash at 70° C. followed by a gradual cooling to room temperature results in peptide 36-dependent cell attachment exclusively in the PIII treated regions of the masked PIII treated sample. These dissociation conditions are required as in the absence of detergent or 70° C. heating the peptide is not efficiently removed from the untreated regions. Additionally if the sample is cooled too rapidly then peptide 36-cell binding activity is not preserved on the PIII treated regions (data not shown). It is worth noting that this patterning is due to a different mechanism to the electrostatic orientation, of peptide 36 on PIII treated polymer at pH 7.4, 150 mM NaCl which sterically hinders cell binding to peptide 36 on the PIII treated regions. Instead this patterning of cells to peptide 36 on the PIII treated regions relies on selective retention of peptide 36. On PIII treated polymer regions the peptide is retained by covalent linkages whist on the untreated regions it is removed. Simultaneous careful selection of buffer and heating conditions allows this retained peptide 36 on the PIII treated regions to bind to cells.

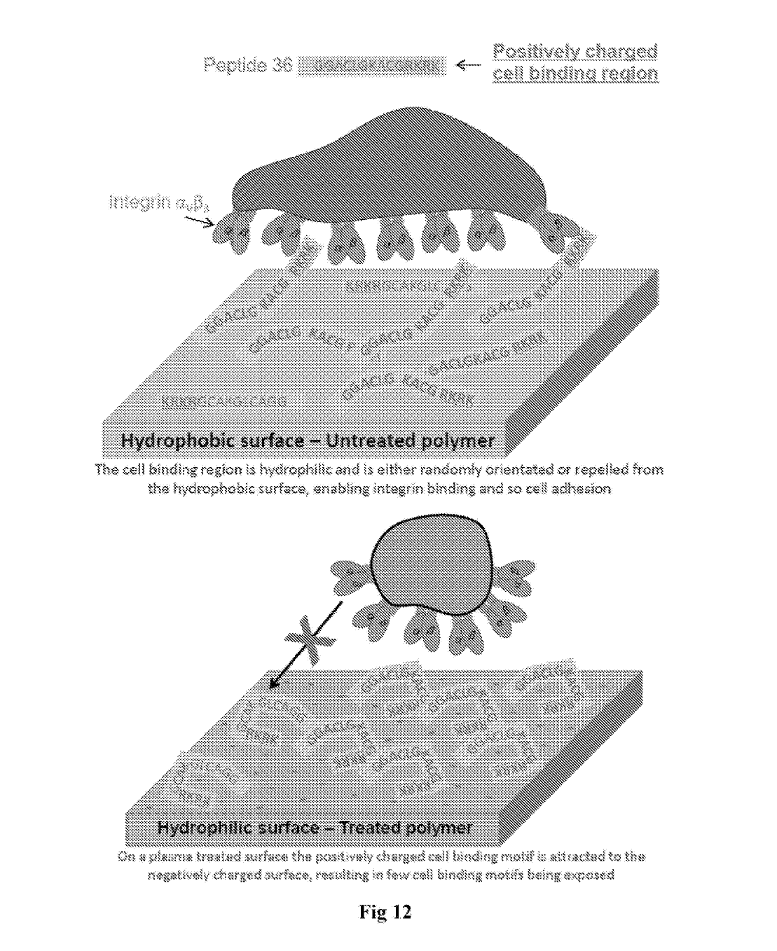

In summary the examples above describe a detailed mechanistic understanding of electrostatic and covalent interactions of a peptide with plasma treated polymers (FIG. 12). Using this understanding we can design conditions to dictate whether this peptide acts to support cell binding on either untreated or PIII treated substrates. We can also use this understanding to design other peptides that can be directly covalently bound to a polymer substrate surface in such a way that the orientation of binding is controlled. This will enable the biologically active sites or motifs of the peptides to be either available for interaction with other agents (e.g. cells, ligands etc.) or masked, as required in specific circumstances.

### REFERENCES

1. Kondyurin A, Nosworthy N J, Bilek M M M. Attachment of horseradish peroxidase to polytetrafluorethylene (teflon) after plasma immersion ion implantation. Acta Biomat 2008; 4(5):1218-1225.
2. Bax D V, McKenzie D R, Weiss A S, Bilek M M M. The linker-free covalent attachment of collagen to plasma immersion ion implantation treated polytetrafluoroethylene and subsequent cell-binding activity. Biomaterials 2010; 31(9):2526-2534.
3. Tiraferri A, Elimelech M. Direct quantification of negatively charged functional groups on membrane surfaces. Journal of Membrane Science 2012; 389:499-508.
4. Wang H, Xu M, Wu Z, Zhang W, Ji J, Chu P K. Biodegradable Poly(Butylene Succinate) Modified by Gas Plasmas and Their In vitro Functions as Bone Implants. ACS Appl Mater Interfaces 2012; 4:4380-4386.
5. Bax D V, Wang Y, Li Z, Maitz P K, McKenzie D R, Bilek M M, et al. Binding of the cell adhesive protein tropoelastin to PTFE through plasma immersion ion implantation treatment. Biomaterials 2011; 32(22):5100-5111.
6. Bax D V, McKenzie D R, Bilek M M M, Weiss A S. Directed cell attachment by tropoelastin on masked plasma immersion ion implantation treated PTFE. Biomaterials 2011; doi:10.1016/j.biomaterials.2011.05.060.
7. Bax D V, Rodgers U R, Bilek M M M, Weiss A S. Cell adhesion to tropoelastin is mediated via the C-terminal GRKRK motif and integrin alpha(V)beta(3). J Biol Chem 2009: 284(42):28616-28623.
8. Buick M M, Bax D V, Kondyurin A, Yin Y, Nosworthy N J, Fisher K, et al. Free radical functionalization of surfaces to prevent adverse responses to biomedical devices. Proc Natl Acad Sci USA August 30; 108(35):14405-14410.
9. Tran C T, Kondyurin A, Chrzanowski W, Bilek M M, McKenzie D R. Influence of pH on yeast immobilization on polystyrene surfaces modified by energetic ion bombardment. Colloids Surf B Biointerfaces April 1; 104:145-152.
10. Tran C T, Kondyurin A, Hirsh S L, McKenzie D R, Bilek M M. Ion-implanted polytetrafluoroethylene enhances Saccharomyces cerevisiae biofilm formation for improved immobilization. J R Soc Interface November 7; 9(76):2923-2935.

1. A method of controlling predominant orientation of direct covalent binding of one or more peptides to a polymer substrate surface comprising:
(a) exposing the surface to energetic ion treatment to generate a plurality of activated sites comprising reactive radical species;
(b) incubating the surface with one or more peptide/s that exhibit or can be induced to exhibit a dipole moment and manipulating the electric field environment and/or charge of said surface and/or of said peptide/s during said incubating;
wherein predominant orientation of direct covalent binding of said peptide/s to said surface is thereby controlled.

2. The method of claim 1 wherein said energetic ion treatment is plasma immersion ion implantation (PIII) or ion beam exposure of an existing polymer substrate surface.

3. The method of claim 1 wherein said energetic ion treatment is energetic ion bombardment during deposition of a plasma polymer.

4. The method of claim 1 wherein the polymer comprises one or more of polyolefins, blends of polyolefins with other polymers or rubber, polyethers, polyamides, polyimides, polycarbonates, halogenated polymers, aromatic polymers, ketone polymers, methacrylate polymers, polyesters and copolymers.

5. (canceled)

6. The method of claim 1 wherein the substrate takes the form of a block, sheet, film, tube, strand, fibre, piece or particle, powder, shaped article, woven fabric or massed fibre pressed into a sheet.

7. The method of claim 1 wherein the polymer comprises a surface of a device.

8. (canceled)

9. The method of claim 7 wherein the device is, or is a component of, a medical device.

10. (canceled)

11. The method of claim 1 further comprising a step of exposing the surface produced in step (a) to oxygen to generate reactive oxygen species at the activated sites.

12. The method of claim 1 wherein said incubating the surface resulting with one or more peptide/s comprises contacting the surface with a solution comprising said one or more peptide/s.

13. The method of claim 12 wherein said manipulating charge of said surface and/or of said peptide/s during said incubating is achieved by controlling pH and/or salt concentration of said solution and/or by applying an external electric field to said solution and/or to said surface.

14. The method of claim 1 wherein said peptides are from about 3 to about 50 amino acids in length.

15. The method of claim 1 wherein within the incubation solution the peptides comprise one or more charged amino acids located at or towards an N and/or C terminus thereof.

16. The method of claim 1 wherein the predominant orientation of peptide/s directly covalent bound to said surface is the orientation of at least about 60% of said peptide/s.

17-19. (canceled)

20. A polymer substrate surface having a peptide directly covalently bound thereto in a manner wherein the predominant orientation of binding of the peptide to the surface is controlled.

21. The polymer substrate surface of claim 20 wherein the polymer comprises one or more of polyolefins, blends of polyolefins with other polymers or rubber, polyethers, polyamides, polyimides, polycarbonates, halogenated polymers, aromatic polymers, ketone polymers, methacrylate polymers, polyesters and copolymers.

22. (canceled)

23. The polymer substrate surface of claim 20 wherein said peptides are from about 3 to about 50 amino acids in length.

24. The polymer substrate surface of claim 20 wherein the predominant orientation of peptide/s directly covalent bound to said surface is the orientation of at least about 60% of said peptide/s.

25-26. (canceled)

27. The polymer substrate surface of claim 20 wherein the substrate takes the form of a block, sheet, film, tube, strand, fibre, piece or particle, powder, shaped article, woven fabric or massed fibre pressed into a sheet.

28. The polymer substrate surface of claim 20 wherein the polymer substrate surface comprises a surface of a device.

29. (canceled)

30. The polymer substrate surface of claim 28 wherein the device is, or is a component of, a medical device.

31. (canceled)

32. A device comprising a polymer substrate surface of claim 20.

33-35. (canceled)
